(19) United States
(12) Patent Application Publication
(10) Pub. No.: US 2026/0315148 A1
(43) Pub. Date: Oct. 15, 2026

(54) SURGICAL PROCEDURE SEGMENTATION

(71) Applicant: Method AI, Inc., Cleveland, OH (US)

(72) Inventors: Charles D. Emery, Gilbert, AZ (US); Jad Kaouk, Hunting Valley, OH (US); Douglas Teany, Medfield, MA (US); John-Michael Sungur, Brookline, MA (US); Jonathan Fincke, Belmont, MA (US); Guy Lavi, Kadima (IL); Adi Dafni, Givat Brenner (IL); Ori Noked, Ramat Gan (IL); Neelima Chavali, Acton, MA (US)

(73) Assignee: Method AI, Inc., Cleveland, OH (US)

(21) Appl. No.: 19/104,537

(22) PCT Filed: Aug. 17, 2023
(86) PCT No.: PCT/US2023/030496

§ 371 Date: Feb. 18, 2025

Related U.S. Application Data
(60) Provisional application No. 63/399,553, filed on Aug. 19, 2022.

(51) Int. Cl. A61B 34/10; A61B 90/00; G06T 7/11; G16H 20/40

(52) U.S. Cl. CPC A61B 34/10; G06T 7/11; G16H 20/40; A61B 2034/107; A61B 2090/378; G06T 2207/10132; G06T 2207/20016; G06T 2207/20081; G06T 2207/20084

(57) ABSTRACT

A trained processor may perform a first segmentation of a non-linear outer surface of a volumetric mass of a targeted feature based upon real-time image data at a first resolution, may define a refinement zone based upon the refinement zone of the non-linear outer surface of the volumetric mass of the targeted feature, and may perform a second segmentation of the non-linear outer surface of the volumetric mass of a targeted feature based upon real-time volumetric ultrasound data within the refinement zone at a second resolution greater than the first resolution.

### CROSS-REFERENCE TO RELATED PATENT APPLICATIONS

This application claims the benefit of U.S. Application No. 63/399,553 entitled SURGICAL PROCEDURE SEGMENTATION filed on Aug. 19, 2022, which is incorporated herein by reference in its entirety.

### BACKGROUND

Image data, such as ultrasound data, is sometimes used to view internal portions of a patient's anatomy during a surgical procedure. Identifying features of interest in ultrasound images, in real-time, presents many challenges.

### BRIEF DESCRIPTION OF THE DRAWINGS

FIG. 1 is a diagram schematically illustrating portions of an example machine learning system for real-time segmentation during a surgical procedure.

FIG. 1A is a diagram illustrating an example real-time ultrasound image with an example overlaid refinement zone for performing higher resolution segmentation to determine a tumor edge.

FIG. 1B₁ is an image of example raw 3D ultrasound imagery of an example tumor mimic.

FIG. 1B₂ is a slice plane/sectional view taken along plane 2 of FIG. 1B₁.

FIG. 1B₃ is a slice plane/sectional view taken along plane 3 of FIG. 1B₁.

FIG. 1B₄ is a slice plane/sectional view taken along plane 4 of FIG. 1B₁.

FIG. 1C₁ is an image of the example raw 3D ultrasound imagery of FIG. 1B₁ further illustrating an example refinement zone.

FIG. 1C₂ is a slice plane/sectional view taken along plane 2 of FIG. 1C₁.

FIG. 1C₃ is a slice plane/sectional view taken along plane 3 of FIG. 1C₁.

FIG. 1C₄ is a slice plane/sectional view taken along plane 4 of FIG. 1C₁.

FIG. 1D₁ is an image of the example raw 3D ultrasound imagery of FIG. 1B₁ further illustrating an example refinement zone.

FIG. 1D₂ is a slice plane/sectional view taken along plane 2 of FIG. 1D₁.

FIG. 1D₃ is a slice plane/sectional view taken along plane 3 of FIG. 1D₁.

FIG. 1D₄ is a slice plane/sectional view taken along plane 4 of FIG. 1D₁.

FIG. 2 is a flow diagram of an example method for training a coarse segmentation network for performing a coarse segmentation on real-time ultrasound image data.

FIG. 3 is a flow diagram of an example method for training a fine segmentation network for performing a fine segmentation in a limited refinement zone on the real-time ultrasound image data.

FIG. 4 is a flow diagram of an example method for a machine trained model to use the coarse segmentation machine learning network and the fine segmentation learning network to segment a clinically relevant targeted feature in a real-time ultrasound image.

FIG. 5 is a diagram schematically illustrating portions of an example machine learning system for real-time segmentation during a surgical procedure.

FIG. 6 is a diagram schematically illustrating portions of an example machine learning system for real-time segmentation during a surgical procedure.

FIG. 7 is a diagram schematically illustrating portions of an example machine learning system for real-time segmentation during a surgical procedure.

FIG. 8 is a diagram schematically illustrating an example of segmentation of real-time ultrasound image data along and about a cutting tool path to a targeted feature within an organ by a machine learning system.

FIG. 9 is a diagram schematically illustrating an example of a determination of a modified cutting tool path based upon the segmentation of FIG. 8 and the determination of path guides along and about the modified cutting tool path.

FIG. 10 is a diagram illustrating an example of a detection of a region using three different example imaging angles.

FIG. 11 is a block diagram schematically illustrating an example extraction system for extracting features from a real-time ultrasound image using a single ultrasound data set.

FIG. 12 is a block diagram schematically illustrating portions of an example extraction system for extracting features from a real-time ultrasound image using multiple ultrasound data sets.

FIG. 13 is a block diagram schematically illustrating portions of an example extraction system for extracting features from multiple sets ultrasound data with different assigned spatial weights.

FIG. 14 is a block diagram schematically illustrating portions of an example extraction system for extracting features from multiple sets of ultrasound data and determining a mean feature extraction.

FIG. 15 is a block diagram schematically illustrating portions of an example extraction system for extracting features from multiple sets of ultrasound data, for applying different spatial weights and for determining a mean feature extraction.

Throughout the drawings, identical reference numbers designate similar, but not necessarily identical, elements. The figures are not necessarily to scale, and the size of some parts may be exaggerated to more clearly illustrate the example shown. Moreover, the drawings provide examples and/or implementations consistent with the description; however, the description is not limited to the examples and/or implementations provided in the drawings.

### DETAILED DESCRIPTION OF EXAMPLES

Disclosed are example machine learning models or machine learning systems that facilitate real-time segmentation of clinically relevant features in image data, such as ultrasound image data, during a surgical procedure. The example systems utilize trained models or trained processor to carry out such real-time segmentation, defining the voxels or coordinates in the real time image that form the outer surface or edge of the clinically relevant features in the ultrasound image. In some implementations, the example systems carry out real-time volume segmentation of a targeted feature or clinically relevant feature in the form of a tumor.

For purposes of this disclosure, the term “ultrasound image” refers to an image or data having characteristics similar to those of a real-time ultrasound image. The term “ultrasound image” may refer to a live or real-time ultrasound image and be designated as such. The term “ultrasound image” may refer to a historic ultrasound image captured by an ultrasound probe or transducer. The term “ultrasound image” may also refer to a synthetic ultrasound image or a versioned ultrasound image. A synthetic ultrasound image may comprise an ultrasound image that is artificially generated using ultrasound principles and predetermined characteristics or derived from observational ultrasound data. For example, a synthetic B-mode ultrasound image may comprise volume data created from a physics-based simulation model (wave equation, speckle simulators, ray tracing, eikonal equation, parabolic equation solvers, geometric (“straight ray” approaches), wherein target characteristics such as the size, shape, orientation, position, sound speed distribution, density distribution and attenuation distribution of the targeted feature of interest, such as a tumor, are varied randomly and/or in a deterministic way that is consistent with the expected characteristic of the target. In some implementations, contrast, resolution, field of view and imaging depth may be fixed or may be varied. The simulated data sets may also include normal anatomy such as vessels, connective tissue, fat, muscle, and other solid organs. The simulations may also include flow features for Doppler imaging.

A synthetic ultrasound image may also comprise an ultrasound image that is artificially generated or created using base, foundational or source images acquired in other modalities. For example, a synthetic ultrasound image may be generated from a computed tomograph (CT) scan or different ultrasound modes as compared to the mode of the real-time ultrasound image. For example, the real-time ultrasound image may be in B-Mode, wherein the synthetic ultrasound images are also in B-Mode, but are derived or generated from A-mode, C-mode, M-mode, Doppler Mode, or other present or future developed ultrasound modes.

A “versioned” ultrasound image refers to an ultrasound image that is been generated from another ultrasound image of the same mode. Multiple “versioned” ultrasound images may be generated for training a machine learning model or network from a single base or source ultrasound image. The versioned ultrasound image may be generated by modifying characteristics of the base or source ultrasound image. For example, the speckle characteristics of a base or source ultrasound image may be modified to produce a second versioned ultrasound image. Likewise, one or more additional ultrasound characteristics may be modified to produce different versions of the base or original ultrasound image. Historic ultrasound images, synthetic ultrasound images and versioned ultrasound images may each be used alone or in combination with one another as part of a larger ultrasound image training set of images for use during training of a machine learning model or network (a trained processor).

For purposes of this disclosure, a machine learning system or network refers to one or more processors that utilize artificial intelligence in that they utilize a network or model that is been trained based upon various source or sample data sets. One example of such a network or model is a fully convolutional neural network. Another example of such a network is a convolutional neural network or other networks having a U-net architecture. Such networks may comprise vision transformers. For example, the model or network may comprise a UNetr transformer, such as those described in Ali Hatamizadeh et al., “UNETR: Transformer for 3D Medical Image Segmentation” (attached as an appendix to this disclosure). Different systems may comprise combinations of different machine learning networks or models.

In some implementations, the determination and identification of the edge of the clinically relevant feature, such as a tumor, is carried out in a multi-step process using multiple segmentation models or networks. For example, a first segmentation model or network, trained using ultrasound images at a first resolution, may be used to determine a coarse estimation of the edges of the feature in the real-time ultrasound image. The first segmentation is performed on down sampled data from the real-time ultrasound image. The use of “down sampled” data means the amount of samples per unit of space and/or time from the real-time ultrasound image that is analyzed to determine a coarse estimation of the edges of the feature is reduced. By contrast, the term “up sampled” or “up sampling” means that the number of samples per unit space and/or time is increased.

For example, the real-time ultrasound image may have a resolution of X numbers of voxels per a unit of volume, Instead of using or analyzing each and every voxel available from the real-time ultrasound image to determine the coarse estimation of the edges of the feature, system 520 uses or samples a predetermined percentage (less than 100%) of the X number of voxels. In some implementations, this “down sampling” may involve using a single predetermined voxel out of every series of consecutive Y voxels in the real-time ultrasound image. For example, the first segmentation model or network may determine the coarse estimation of the edges of the feature using every other voxel (where Y equals two) in a series of consecutive voxels, every third voxel (where Y equals three) in a series of consecutive voxels or every Mth voxel (where Y equals M) in a series of consecutive voxels. Such down sampling may occur across the entire real-time ultrasound image or may occur within a smaller predefined portion of the real-time ultrasound image.

This rough or coarse estimation may then be used to define a refinement zone. The refinement zone is an area, smaller than the entire real-time ultrasound image, where the actual edge of the targeted feature is expected to lie. The refinement zone may have an inner boundary and an outer boundary, wherein edges of the targeted feature are expected to lie between the inner boundary and the outer boundary. In some implementations, the inner boundary of the refinement zone may be determined by deflating the coarse segmentation edge. For example, the inner boundary may be inwardly spaced from the coarse segmentation edge by a predetermined distance (number of pixels or voxels). In such implementations, the outer boundary of the refinement zone a be determined by inflating the coarse segmentation edge. For example, the outer boundary may be outwardly spaced from the coarse segmentation edge by predetermined distance (number of pixels or voxels).

In some implementations, the refinement zone may be directly input by a physician or healthcare worker. For example, a physician may move a cursor or stylus along a screen to digitally draw inner and outer boundaries of the refinement zone about the coarse segmentation edge. In some implementations, the establishment of the refinement zone for carrying out the finer, more precise feature edge segmentation may be performed without reliance upon an earlier coarse feature edge segmentation. For example, in some implementations, the physician may move a cursor or draw with a stylus identifying those portions of the live ultrasound image for which the finer feature edge segmentation at the high-resolution may be performed.

In some implementations, a machine trained model or network may be used to identify or determine the inner and outer boundaries of the refinement zone. For example, a network may be trained using different ultrasound images, each ultrasound training image including an initial feature edge segmentation and inner and outer boundaries of a refinement zone about the initial feature edge segmentation. The network may apply inner and outer boundaries to the real-time ultrasound image based upon the coarse feature edge segmentation in the real-time ultrasound image.

In some implementations, a coarse network may be trained using different ultrasound images, each ultrasound training image including just the inner and outer boundaries of a refinement zone and not including any prior target segmentation. The network may generate inner and outer boundaries to the real-time ultrasound image based upon such training.

A second segmentation model or network, trained using ultrasound images, is performed for those pixels/voxels of the real-time ultrasound image within the refinement zone to determine more refined, more precisely located coordinates of the edges of the feature. The location(s) could be learned using regression methods or segmentation techniques which rely on computing the probability that a given pixel or pixel cluster contains tumor or some other tissue or object.

In those implementations where an initial coarse feature edge segmentation is performed, the second segmentation model or network may be trained with ultrasound images at a second resolution, greater than the first resolution of the training images used to train the first network for performing first segmentation. The second segmentation model or network may be trained using ultrasound images at a second resolution that corresponds to the resolution at which the data or voxels in the refinement zone are to be analyzed to determine the more refined, more precisely located coordinates of the edges of the feature.

In some implementations, the second segmentation model or network may sample voxel data within the refinement zone of the real-time ultrasound image at a rate (resolution) that is greater than the first resolution used to determine the coarse estimation of the feature edges but less than the actual resolution of the real-time ultrasound image. For example, the first segmentation model or network may sample a first percentage of voxel data across the entirety of or within a predefined region of the real-time ultrasound image while the second segmentation model or network may use or sample a second percentage, greater than the first percentage, but less than 100%, of the voxel data within the refinement zone of the real-time ultrasound image

In some implementations, the second segmentation model or network may sample voxel data within the refinement zone of the real-time ultrasound image at a rate (resolution) that corresponds to or is equal to the resolution of the real-time ultrasound image. For example, the first segmentation model or network may sample or use a first percentage (less than 100%) of voxel data across the entirety of or within a predefined region of the real-time ultrasound image while the second segmentation model or network uses 100% of the voxel data within the refinement zone from the real-time ultrasound image.

In some implementations, the second segmentation model or network may sample voxel data within the refinement zone from the real-time ultrasound image at an up sampled rate (resolution). For example, the first segmentation may sample or use a first percentage (less than 100 percent) of voxel data across the entirety of within a predefined region of the real-time ultrasound image, wherein the second segmentation model or network up samples the voxel data within the refinement zone of the real-time ultrasound image, using a number of samples per unit time that exceeds the original sampling of the real-time ultrasound image.

Because the second segmentation is carried out using a second network trained at a higher resolution in the refinement zone of the real-time ultrasound image (analyzing a greater number of pixels/voxels per area or volume), the resolution of the estimated edge is enhanced. At the same time, because the second segmentation is applied to just the refinement zone, the total number of voxels being analyzed is reduced (as compared to analyzing every voxel across the entire real-time ultrasound image at the higher resolution), reducing computational time and permitting the overall segmentation of the feature to be more likely performed in real-time or with less processing resources.

Although the course segmentation to identify the refinement zone and the second segmentation in the refinement zone are described as being performed by two networks, in other implementations, the segmentations may alternatively be performed by a single network or more than two networks.

The disclosed example machine learning systems may further generate or determine and apply surgical guidance guard rails to the real-time ultrasound image. Such guard rails may include an inner guard rail and an outer guard rail. The guard rails serve as boundaries for guiding the path or trajectory of a surgical tool, such as a cutting tool. With respect to the removal of a tumor, the inner guard rail defines an inner-most boundary for the cutting path that provides a satisfactory degree of confidence that the entire tumor will be cut away and removed. In other words, if the cutting tool path intercepts the inner boundary and moves inward of the inner boundary, there is a greater chance that the entirety of the tumor may not be removed. The outer guard rail defines an outer most boundary for the cutting path that attempts to ensure that the entire tumor will be cut away while also reducing or minimizing the removal of otherwise healthy features or tissue. In other words, if the cutting tool path intercepts the outer boundary and moves outward of the outer boundary, otherwise healthy tissue may be unnecessarily cut or removed.

In some implementations, the inner guard rail and the outer guard rail are based upon an earlier segmented edge of a targeted feature, such as the estimated edge of a tumor. In other words, the inner guard rail and the outer guard rail are determined based upon the estimated outer surface of the feature, such as the outer surface of a tumor. In some implementations, the inner guard rail and the outer guard rail may be based upon the above-described coarse or rough segmentation, wherein the second finer segmentation may or may not be performed. In some implementations, the inner guard rail and the outer guard rail may be based upon the second higher resolution feature edge segmentation. In some implementations, the inner guard rail may coincide with the coarse or finer feature edge segmentation. In some implementations, the first, coarse feature edge segmentation or the second finer feature edge segmentation may be inflated by a first amount to define the inner guard rail. In some implementations, the first coarse feature segmentation or the second finer feature edge segmentation may be inflated by a second greater amount to define the outer guard rail. Inflation of a feature edge segmentation refers to the uniform outward movement or spacing of the feature edge segmentation along the perimeter of the feature edge segmentation.

In some implementations, the inner and outer guard rails may be determined using a machine learning model or network trained using ultrasound training images that include a designated training inner guard rail and a designated training outer guard rail. In some implementations, the inner guard rail may be defined are determined based upon a prior feature edge segmentation (coarse or rough), wherein the outer guard rail is differently determined, such as through a physician's selection or input or based upon a machine trained model or network trained using ultrasound images that include a designated outer guard rail.

In particular implementations, the machine learning system may identify the edges or presence of various non-targeted features in the real-time ultrasound image by segmenting such non-targeted features. Such non-targeted features are those anatomical features, the presence of which within or proximate to cutting path or other path of the surgical tool, may impair the performance of the surgical procedure or its result. Examples of such non-targeted features include, but are not limited to, veins, arteries and nerves bundles. Stated another way, the non-targeted features are features that are not targeted for treatment and/or removal. In one implementation a tumor to be excised is the targeted feature and nerves or arteries are not targeted for removal but removal of which may impair the functioning of the organ.

In some implementations, the machine learning system may carry out such segmentation of non-targeted features in selected portions of the real-time ultrasound image to reduce computational load and to maintain real-time data regarding such non-targeted features. For example, in some implementations, the example machine learning system may correlate non-targeted features to those pixels or regions of the real-time ultrasound image contained within a segmentation buffer zone. In some implementations, the segmentation buffer zone may coincide with the inner and outer guard rails described above. In some implementations, the segmentation buffer zone may include an inner boundary coinciding with the coarse or refined targeted feature edge segmentation, such as the estimated edge of the tumor, wherein the outer boundary coincides with the outer guard rail. In some implementations, the segmentation buffer zone may extend beyond the outer guard rail, by a predetermined distance, to encompass any non-targeted features that may be sufficiently close the guard rail so as to warrant special precautions when controlling movement of the surgical tool along its path between the guard rails.

In particular implementations, the segmentation of the non-targeted features may be carried out in a multi-step or staged process. For example, a coarse or rough segmentation aimed at identifying such non-targeted features may be performed using a first machine trained model or network that is been trained based upon ultrasound images at a first resolution depicting the non-targeted feature of interest. This segmentation may be carried out for all those pixels/voxels lying within the segmentation buffer zone or may be carried out on a down sampled set of data from the segmentation buffer zone.

Thereafter, the coarse segmented non-targeted feature coordinates may be used as a basis for defining a smaller non-targeted feature refinement zone about the non-targeted feature. Similar to the refinement zone used to refine the estimated location of the edge of the targeted feature, such as the edge or outer surface of the tumor, the smaller non-targeted feature refinement zone may be used to refine the coordinates, size, orientation or the like of the non-targeted feature. In particular, a second machine trained model or network, trained based upon ultrasound images at a second resolution, greater than the first resolution, and depicting the non-targeted feature of interest may be used. The second machine trained model or network may more precisely define the edges of the non-targeted feature to more precisely define its location. Because the second non-targeted feature segmentation is carried out using a second network trained at a higher resolution and employing higher resolution of data sampling in the non-targeted feature refinement zone of the real-time ultrasound image (analyzing a greater number of voxels per area or volume), the resolution of the estimated edge of the non-targeted feature is enhanced. At the same time, because the second non-target feature segmentation is applied to just the non-targeted feature refinement zone, the total number of voxels being analyzed is reduced, reducing computational time and permitting the overall segmentation of the feature to be more likely performed in real-time or with less processing resources.

In some implementations, the processor of the example machine learning systems may output a warning or notification to a physician or other healthcare worker indicating the presence of a non-targeted feature within or proximate to the guard rails. In some implementations, the system may perform a multi-class segmentation of the non-targeted feature. For example, the system may identify the non-targeted segmented feature as a nerve bundle, a vein or an artery. In some implementations, the classification may be carried out by a machine learning network trained to identify different classifications of non-targeted features in an ultrasound image. In some implementations, the warning or notification may be varied depending upon the classification of the non-targeted feature, its size, its location relative to the inner guard rail, its location relative to the outer guard rail, and/or a loss in organ or patient anatomical functioning should the non-targeted feature be severed or damaged. For example, different visual, audible and/or haptic warnings may be output based on the classification of the non-targeted feature, its size, its location relative to the inner guard rail, its location relative to the outer guard rail, and/or a loss in organ or patient anatomical functioning should the non-targeted feature be severed or damaged, The intensity of the warning (brightness, loudness, amplitude and/or frequency of the notice) may vary based on a determined severity of the circumstance. Satisfaction of different thresholds may trigger different notice “intensities and/or different notice modalities/mechanisms.

In particular implementations, the processor of the example machine learning system may further restrict movement of a surgical tool, such as a cutting tool, along its path within the inner and outer guard rails based upon the determined presence of a non-targeted feature between the guard rails or proximate to a guard rail. In some implementations, the coordinates of the inner guard rail and/or the outer guard rail may be adjusted, bent inwardly or bent outwardly at particular locations or portions about the targeted feature, (the tumor) to more tightly restrict or control the available area for the cutting path in regions proximate to the identified location of the non-targeted feature.

For purposes of this application, the term “processing unit” shall mean a presently developed or future developed computing hardware that executes sequences of instructions contained in a non-transitory memory. Execution of the sequences of instructions causes the processing unit to perform steps such as generating control signals. The instructions may be loaded in a random-access memory (RAM) for execution by the processing unit from a read only memory (ROM), a mass storage device, or some other persistent storage. In other embodiments, hard wired circuitry may be used in place of or in combination with software instructions to implement the functions described. For example, a controller may be embodied as part of one or more application-specific integrated circuits (ASICs). Unless otherwise specifically noted, the controller is not limited to any specific combination of hardware circuitry and software, nor to any particular source for the instructions executed by the processing unit.

For purposes of this disclosure, the term “coupled” shall mean the joining of two members directly or indirectly to one another. Such joining may be stationary in nature or movable in nature. Such joining may be achieved with the two members, or the two members and any additional intermediate members being integrally formed as a single unitary body with one another or with the two members or the two members and any additional intermediate member being attached to one another. Such joining may be permanent in nature or alternatively may be removable or releasable in nature.

For purposes of this disclosure, the phrase “configured to” denotes an actual state of configuration that fundamentally ties the stated function/use to the physical characteristics of the feature proceeding the phrase “configured to”.

For purposes of this disclosure, unless explicitly recited to the contrary, the determination of something “based on” or “based upon” certain information or factors means that the determination is made as a result of or using at least such information or factors; it does not necessarily mean that the determination is made solely using such information or factors. For purposes of this disclosure, unless explicitly recited to the contrary, an action or response “based on” or “based upon” certain information or factors means that the action is in response to or as a result of such information or factors; it does not necessarily mean that the action results solely in response to such information or factors.

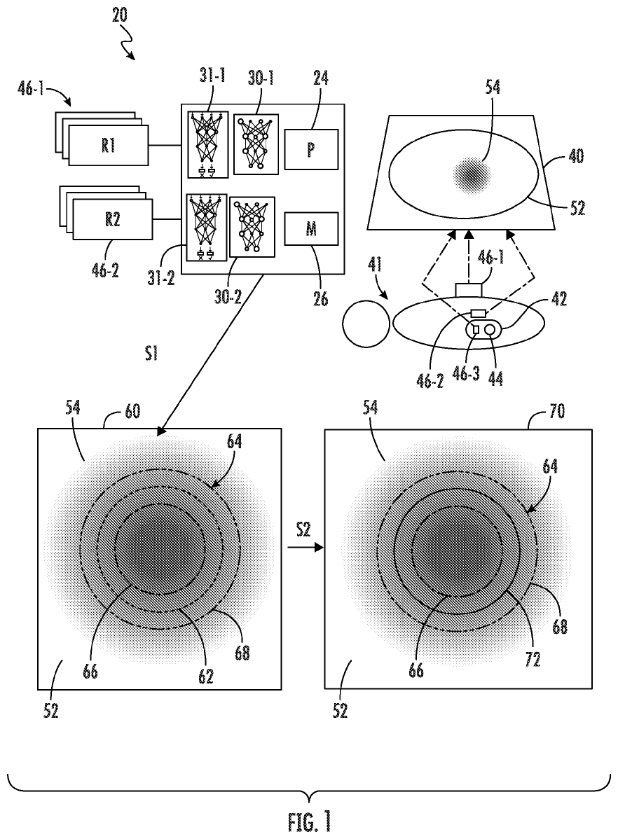

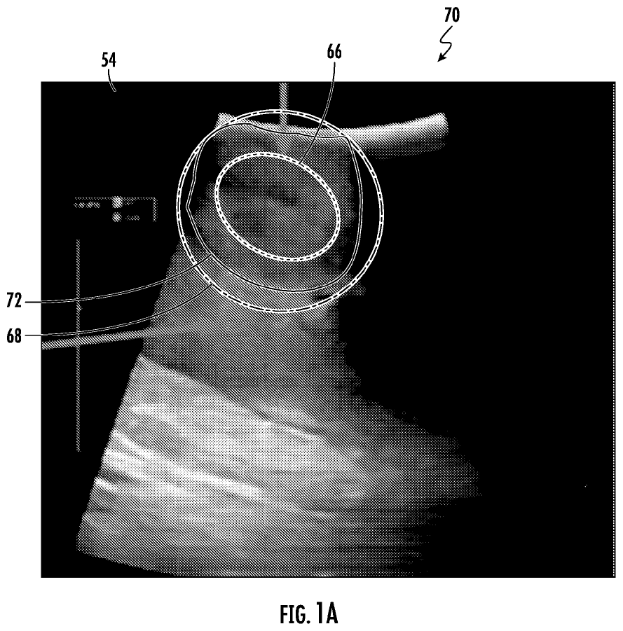

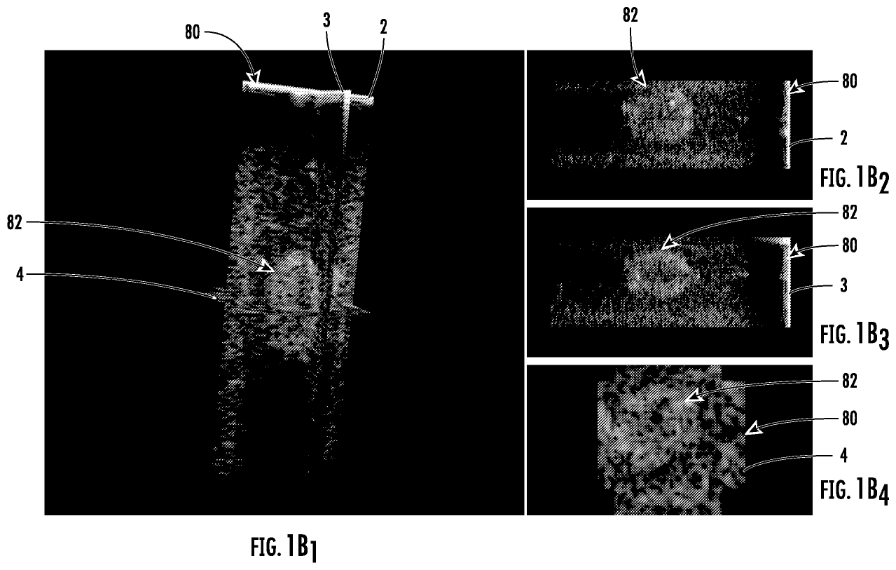

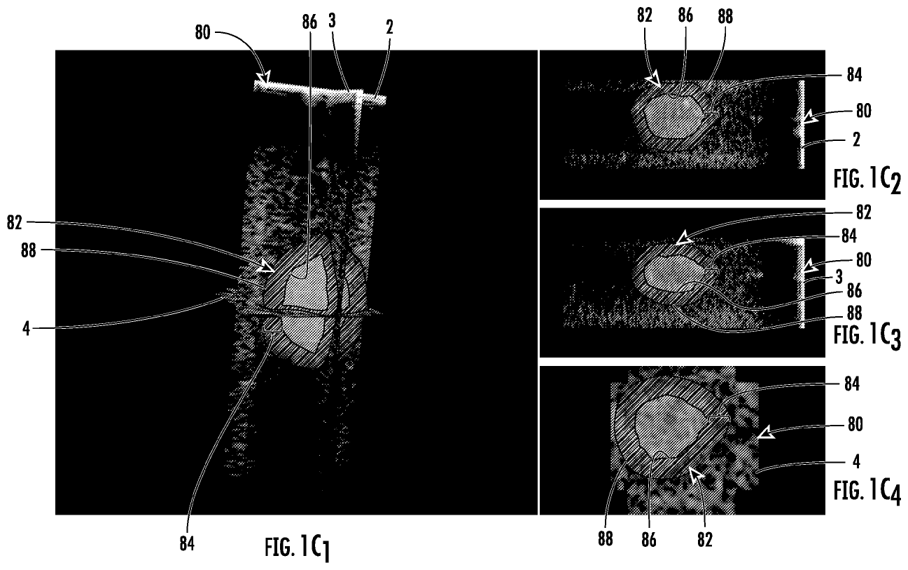

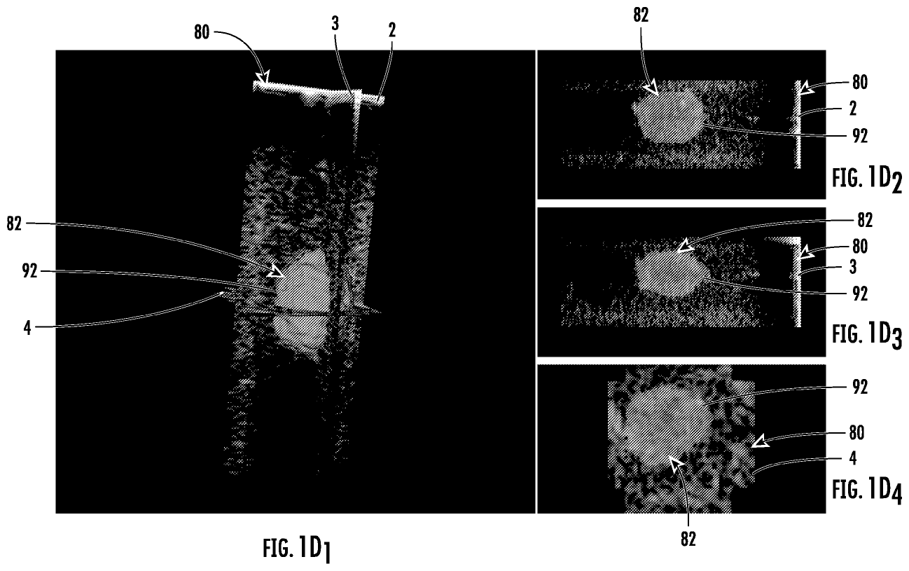

FIG. 1 is a diagram schematically illustrating portions of an example machine learning system 20 for real-time volumetric segmentation of clinically relevant features during a surgical procedure. System 20 employs a trained processor 24, memory 26 and machine learning models or networks 30-1, 30-2 (collectively referred to as networks 30) to carry out such segmentation, defining the pixels or coordinates in the real-time image that form the outer surface or edge of the clinically relevant features in the ultrasound image.

FIG. 1 illustrates a real-time or live ultrasound image 40 of an anatomy of a patient 41 including an internal organ 42 (such as a kidney) having a clinically relevant feature or targeted feature 44 (shown as a suspected tumor). Ultrasound sensor 46 (shown as sensors 46-1, 46-2 and/or 46-3) may comprise one or more sensors that are configured to capture different volumetric real-time ultrasound images of organ 42 and the feature of interest, tumor 44. In the example illustrated, ultrasound image 40 comprises a B-mode ultrasound image. The B-mode ultrasound image is in real-time and includes ultrasound signals or data that correspond to a depiction 52 of the organ 42 and a depiction 54 of tumor 44. In the ultrasound image 40, depiction 54 of the targeted feature such as tumor 44 may not include well-defined edges or boundaries. Such edges or boundaries may be further obfuscated by noise and speckle. In other implementations, other organs or clinically relevant features or targeted features may likewise be sensed and imaged by system 20. In other implementations, the live or real-time ultrasound image 40 may comprise other forms of ultrasound images, such as other ultrasound image modes or data types, such as Doppler, elastography, acoustic radiation force imaging (ARFI), and compound imaging (frequency or spatial), strain, tomographic, velocity, attenuation, opto-acoustic, and density.

In the example illustrated, ultrasound probe 42-1 comprise a surface probe positioned on the exterior of the patient 41. Ultrasound probe 42-2 comprises an organ surface ultrasound probe either laparoscopically positioned on the exterior surface of organ 42 or inserted through a trocar or cannula into patient 41 and position and retained along the surface of organ 42. Ultrasound probe 42-3 may comprise a micro-transducer which is inserted into an interior of the organ 42. In some implementations, the real-time ultrasound image 40 may be produced or generated in real-time by any of the ultrasound probes 46.

The term real-time as used herein means providing updated information with such frequency that a user does not notice any delay and perceives the feedback as instantaneous. State another way, images or other information are refreshed with sufficient frequency to provide a surgeon with current information as the surgeon manipulates a robotic tool in a surgical arena. With respect to real-time display of ultrasound images includes providing multiple images of an anatomical structure in the form of motion. In one example the display is updated multiple times per second. In one example the displayed image is updated every 500 ms or less. In one example the display is updated with over 10 images per second. In another example the display is updated with over 20 images images per second.

Trained processor 24 comprises a processing unit configured to carry out instructions contained in memory 26. Memory 26 comprise a non-transitory computerized readable medium containing instructions for directing processor 24 to perform segmentation of the data received and corresponding to ultrasound image 40 using networks 30. Network 30-1 comprises a machine trained model or network created during a training phase of an in-training network 31-1 using or based upon a set of ultrasound images 46-1 having a first coarse or lower resolution R1. The model or network 30-1 performs multiple iterations with the training data set of ultrasound images 46-1 to learn those particular features or characteristics corresponding to the clinically relevant feature, such as a tumor edge. The model or network 30-1 may further learn those particular features or characteristics that are not associated with the clinically relevant feature, such as healthy tissue about a tumor edge.

As shown by display 60, which illustrates an enlarged portion of the image 40, processor 24 applies the machine trained model network 30-1 to the data from the live or real-time ultrasound image 40 to perform a first coarse, low resolution, segmentation of the volumetric data provided by image 40 to infer or estimate an edge or outer surface 62 of the depiction 54 corresponding to the actual outer edge surface of the tumor 44. The data corresponding to the real-time ultrasound image 40 is down sampled to match or closely approximate the resolution R1 of the training set of ultrasound images 46-1 used to train network 30-1. The segmentation or outer surface 62 will likewise have a resolution R1 corresponding to the resolution of the training set of ultrasound images 46-1 used to train network 30-1. The down sampling of data from image 40 and the use of the training set of ultrasound images 46 1 at the resolution R1 reduces the overall number of pixels that are processed and input into network 30-1, reducing the processing time for performing this first coarse segmentation. In one implementation outer surface 62 of tumor 44 is a non-linear shape. FIG. 1 is a schematic representation of a two-dimensional view of tumor 44 illustrating outer surface 62 as having a circular shape. In reality, outer surface 62 will have a three-dimensional shape that approximately matches the actual outer surface of the non-linear shape. While the shape of surface 62 may be curvilinear it will not have a constant radius unless of course the actual tumor surface has a constant radius. Said another way, the tumor may have an arbitrary or an amorphous shape.

As further shown by display 60, processor 24, following instructions contained in memory 26, may use the results of the first segmentation, the coarse estimate for the outer surface 62, as a basis for determining a refinement zone 64. The refinement zone 64 is an area, smaller than the entire real-time ultrasound image 40, where the actual edge of the targeted feature 44 is expected to lie. The refinement zone 64 may have an inner boundary 66 and an outer boundary 68, wherein data corresponding to edges of the targeted feature 44 are expected to lie between the inner boundary 66 and the outer boundary 68. In some implementations, the inner boundary 66 of the refinement zone 64 may be determined by deflating the coarse segmentation edge 62. For example, the inner boundary 66 may be inwardly spaced from the coarse segmentation edge 62 by a predetermined distance (number of pixels). In such implementations, the outer boundary 68 of the refinement zone 64 a be determined by inflating the coarse segmentation edge 62. For example, the outer boundary may be outwardly spaced from the coarse segmentation edge's 62 by predetermined distance (number of pixels).

Processor 24, following instructions contained in memory 26, utilizes the refinement zone 64 to select those regions or portions of ultrasound image 40 for performing a second volumetric segmentation (S2) at a higher resolution to estimate a more precise outer surface of the targeted feature, the tumor 44. As shown by display 70, which illustrates an enlarged portion of the image 40 following the second segmentation at the higher resolution. To perform the second segmentation at the high-resolution, processor 24 applies the machine trained model network 30-2 to the data from the live or real-time ultrasound image 40 to the data or pixels within refinement zone 64. The data corresponding to the real-time ultrasound image 40 within refinement zone 64 is sampled at a rate to match or closely approximate the resolution R2 of the training set of ultrasound images 46-2 used to train network 30-1. Processor 24 uses network 30-2 to perform the second refined segmentation of the volumetric data provided by image 40 to infer or estimate an edge or outer surface 72 of the depiction 54 corresponding to the actual outer edge surface of the tumor 44. The segmentation or outer surface 72 will likewise have a resolution R2 corresponding to the resolution of the training set of ultrasound images 46-2 used to train network 30-2. The up sampling of data from image 40 (relative to down sampling of the data for the first coarse segmentation) and the use of the training set of ultrasound images 46-2 at the resolution R2 increases the precision of the estimated outer surface location for tumor 44, facilitating more accurate guidance for a cutting tool path proximate to the outer surface of the tumor 44. At the same time, because the higher resolution segmentation which analyzes a greater percentage or number of pixels is limited to those regions within the refinement zone 64, the overall number of pixels that are processed and input into network 30-2 may be reduced, reducing the processing time for performing this second or refined segmentation of the outer surface.

FIG. 1A is a diagram illustrating an example real-time ultrasound image with an example overlaid refinement zone for performing higher resolution segmentation to determine a tumor edge. The example refinement zone has an inner boundary 66 and an outer boundary 68 with the higher resolution segmented tumor boundary 72.

FIG. 1B₁ is an image 80 of example raw 3D ultrasound imagery of an example tumor mimic 82 captured by at least one of sensors 46 of system 20. FIG. 1B₂ is a slice plane/sectional view taken along plane 2 of FIG. 1B₁. FIG. 1B₃ is a slice plane/sectional view taken along plane 3 of FIG. 1B₁. FIG. 1B₄ is a slice plane/sectional view taken along plane 4 of FIG. 1B₁.

FIG. 1C₁ is the image 80 of the example raw 3D ultrasound imagery of FIG. 1B₁ further illustrating an example refinement zone 84 having an inner boundary 86 and an outer boundary 88. FIG. 1C2 is a slice plane/sectional view taken along plane 2 of FIG. 1C1. FIG. 1C3 is a slice plane/sectional view taken along plane 3 of FIG. 1C1. FIG. 1C4 is a slice plane/sectional view taken along plane 4 of FIG. 1C1. Inner boundary 86 and outer boundary 88 of refinement zone 84 may be determined by system 20 in a manner similar to the determination of boundaries 66 and 68 of refinement zone 64 described above.

FIG. 1D₁ is the image 80 of the example raw 3D ultrasound imagery of FIG. 1B₁ further illustrating an example segmentation of the example tumor mimic 82. The segmentation results in the identification of a more precise boundary 92 of the tumor 82 which may enhance surgical procedures, such as tumor excision.

FIG. 1D₂ is a slice plane/sectional view taken along plane 2 of FIG. 1D₁. FIG. 1D₃ is a slice plane/sectional view taken along plane 3 of FIG. 1D₁. FIG. 1D₄ is a slice plane/sectional view taken along plane 4 of FIG. 1D₁.

The segmentation may be performed in a manner similar to the segmentation described above with respect to FIGS. 1 and 1A. As with the segmentation shown in FIGS. 1 and 1A, the segmentation shown in FIG. 1D₁ may be performed within the refinement zone 84 shown in FIG. 1C₁. The segmentation may be at a second resolution that is greater than the segmentation used to determine the inner and outer boundaries 86 and 88. Although each of the above FIGS. 1B, 1C, and 1D use orthogonal axes, in other implementations they can be rendered in other coordinate systems, such as polar, cylindrical, spherical and non-orthogonal coordinate systems, as advantageous to the user or application. Although each of the above planes 2, 3 and 4 are illustrated at a particular location along an axis orthogonal to such planes, it should be appreciated that the planes may each be individually moved to different locations along their respective orthogonal axes.

FIG. 2 is a flow diagram illustrating an example method 100 that may be used to train a model or network 30-1 of system 20. In other implementations, network 30-1 may have other configurations and may be trained in other fashions in other implementations. As indicated by block 104, a coarse network 30-1 such as UNETR receives volume data in the form of ultrasound training data which may be comprised of volumetric imagery, numerous 2D images, numerous 2D images with a known spatial relationship and volumetric ultrasound data that may tapped anywhere along the ultrasound image formation chain (element data, RF data, beamformed RF data, detected beamformed RF data, line data and final imagery) 46-1. Training data 46-1 comprises a set of ultrasound data at a first coarse resolution R1. As discussed above, the training images 46-1 may comprise historic ultrasound data, synthetic ultrasound images, versioned ultrasound images, or combinations thereof.

As indicated by block 106, the coarse training network 31-1 outputs the coarse estimate of the volume/shell coordinates the from the outer surface coordinates) of the targeted feature, such as the outer surface of the tumor 44. As indicated by block 108, processor 24 computes an error metric based upon ground truth segmentation. As indicated by arrow/block 110, the error metric is back propagated to the coarse in training network 31-1 and model weights are updated. This process is iteratively repeated until a coarse model or network 30-1 with satisfactory error values is determined.

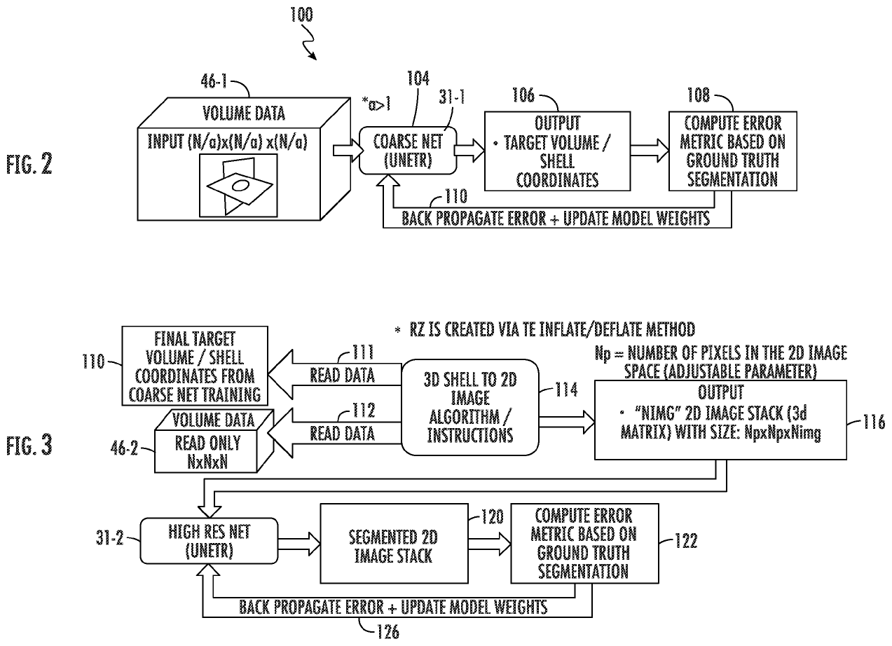

FIG. 3 is a flow diagram illustrating an example method that may be used to train a machine learning model or network 30-2. In other implementations network 30-2 may have other configurations and such or may be trained in other fashions. As indicated by block 114, a 3D to 2D image algorithm/instructions contained in memory 26 directs the processor 24 to read the final coarse volume/shell (outer surface) coordinates 110 of the tumor 44 (as indicated by arrow 111). Based upon such coordinates, processor 24 carries out a deflation of the coordinates to determine the inner boundary 66 of the training refinement zone 64 and inflates the coordinates to determine an outer boundary 68 of training refinement zone 64. As indicated by arrow 112, the instructions 114 further direct the processor 24 to read the volume data in the set 46-2, but only in the training refinement zone 64. Training images 46-2 comprises the same set of ultrasound training images forming set of training images 46-1, but at a second greater resolution R2. As discussed above, the training images 46-1 (and 46-2) may comprise historic ultrasound images, synthetic ultrasound images, versioned ultrasound images, or combinations thereof.

As indicated by block 116, processor 24, following instructions 114, outputs a 2D image stack (3D matrix), the instructions in 114 perform an operation that converts volume data to 2D data, the conversion could be executed using projection methods as azimuthal, cylindrical and conical techniques. The 2D image stack is provided to the high-resolution in training network 31-2 for training the network to form the final network 30-2. In the example illustrated, high-resolution networks 30-1 and 30-2 are each a UNETR network. In other implementations, the high-resolution in machine learning network 30-1 and the machine learning network 30-2 may comprise other networks, such as convolutional neural networks or the like.

As indicated by block 120, the high-resolution in training network 31-2 carries out the second segmentation on the volume data within the refinement zone (read in block/arrow 112), the 2D image stack. As indicated by block 122, processor 24 computes an error metric for the results of block 120 based upon ground truth segmentation. As indicated by block 126, the error metric is back propagated to network 31-2 and model weights are updated. This process are iteratively repeated until a final model or network 30-2 with satisfactory error values is generated. In some implementations, blocks 110-116 could be executed before training to create a data set that is then used to train 31-2 or the 110-116 could be executed in batches during training.

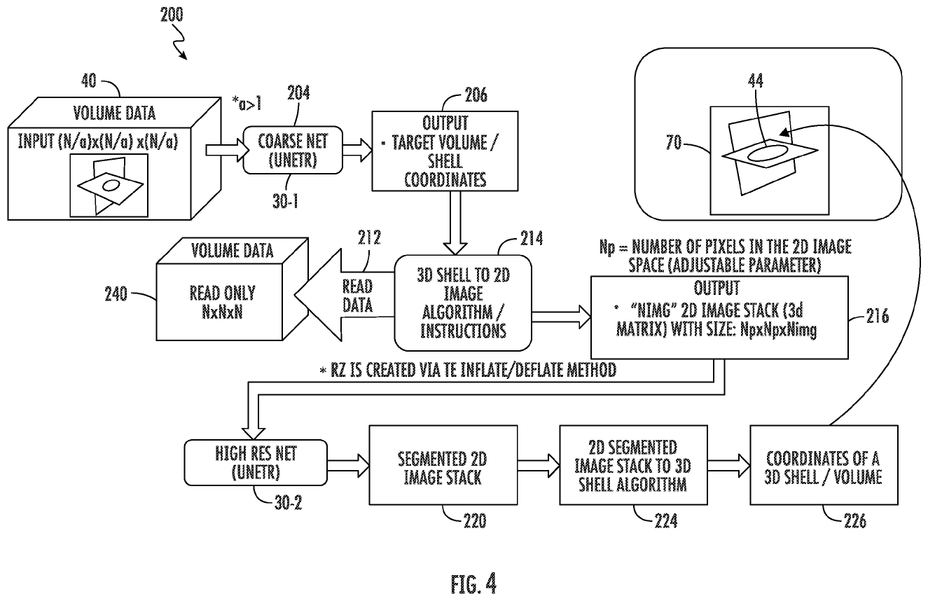

FIG. 4 is a flow diagram of an example method 200 for inferencing or estimating the coordinates of the outer surface of the clinical feature of interest, such as tumor 44, in a real-time ultrasound image 40 using the machine trained models or networks 30-1 and 30-2 (trained as described above). As indicated by block 204, the coarse model or network 30-1 reads or receives volume data from the real-time ultrasound image. As indicated by block 206, network 30-1 outputs coarse coordinates for the target volume/shell (outer surface).

As indicated by block 214, 3D shell to 2D image algorithm/instructions contained in memory 26 direct processor 24 to determine the real-time refinement zone 64 in the real-time ultrasound image by deflating the coarse shell coordinates determined in block 206 to determine the inner boundary 66 and by inflating the coarse shell coordinates determined in block 206 to determine the outer boundary 68 of the real-time refinement zone 64. As indicated by arrow 212, the instructions contained in memory 26 further direct processor 24 to read volume data from the real-time ultrasound volume 40, but only in the real-time refinement zone 64. This volume data may be up sampled relative to the sampling rate used during the coarse segmentation.

As indicated by block 216, processor 24, following instructions 114, outputs a NIMg 2D image stack (3D matrix). The 2D image stack is provided to the high-resolution network 30-2. In the example illustrated, high-resolution network is a UNETR network. In other implementations, the high-resolution network 30-2 may comprise other networks, such as convolutional neural networks or the like.

As indicated by block 220, the high-resolution network 30-2 carries out the second segmentation on the volume data within the refinement zone (read in arrow 212), the 2D image stack. As indicated by block 224, the second segmentation, at the high-resolution, results in a 2D segmented image stack. This image stack is provided to a 3D shell algorithm (stored in memory 26 and carried out by processor 24). As indicated by block 226, processor 24 carries out the 3D shell algorithm to output the coordinates of a 3D shell/volume (outer surface) as depicted in the display 70 of FIG. 4, depicting the example real-time ultrasound image 40 with the volumetric segmentation of the outer surface of the targeted feature, the tumor 44. In one implementation, the first network 30-1 yields sufficient segmentation for an intended purpose such that a second refinement segmentation is not required.

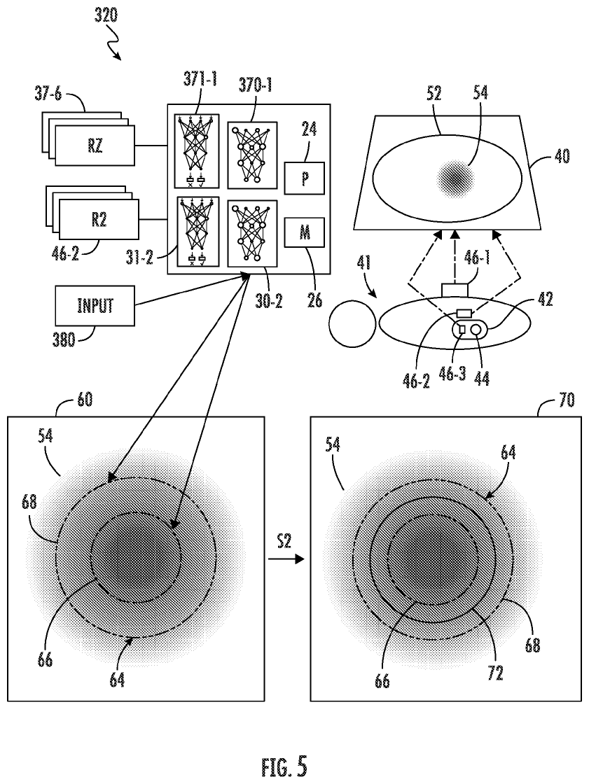

FIG. 5 is a diagram schematically illustrating portions of an example machine learning system 320. FIG. 5 illustrates other examples of how a refinement zone may be determined to segment the outer surface of the clinically relevant features, a targeted feature, such as a tumor. System 320 is similar to system 20 described above except that, in addition to offering a mode where the refinement zone 64 is determined by inflating and deflating initial coarse estimates as described above, system 320 offers additional alternative user selectable modes for determining the refinement zone 64. Those components of system 320 that correspond to components of system 20 are numbered similarly and/or are shown in and described with respect to FIG. 1.

FIG. 5 illustrates two alternative user selectable modes for determining refinement zone 64 which is used as described above in FIGS. 1-4 to reduce the regions of a live ultrasound image that are processed when segmenting a targeted feature in a real-time ultrasound image. In a first additional mode, system 320 may determine refinement zone 64 using a network. In the example illustrated, network 370-1 comprises a machine learning model or network created during a training phase or mode of an in-training network 371-1 using or based upon a set of training ultrasound images 376 which each comprise data corresponding to the targeted feature and the coordinates of the inner and outer boundaries of a training refinement zone. The in-training network 371-1 performs multiple iterations with the training data set of ultrasound images 376 to learn those particular features or characteristics which are determinative of where the refinement zone should be located relative to the targeted feature. The set of training images 376 may comprise historical ultrasound images, synthetic ultrasound images, versioned ultrasound images, or combinations thereof. The training of network 370-1 may be performed in a fashion similar to that described above with respect to the training of network 30-1 as described above with respect to FIG. 2.

The refinement zone 64 in the real-time ultrasound image 40 is determined by analyzing ultrasound data of the real-time ultrasound image 40 using network 370-1 to determine the real-time refinement zone 64 shown in display 60. In the example illustrated, the ultrasound data comprises volumetric data. In other implementations, other forms of ultrasound data, whether static data or data over time, may be analyzed to determine the refinement zone. Examples of such other data include, but are not limited to two-dimensional B-Mode data, doppler data, and elastography data.

Thereafter, the ultrasound data from only within the refinement zone 64 is processed or analyzed using network 30-2 to carry out the second segmentation result in the refined higher resolution estimation for the outer surface 72 shown in display 70. In some implementations, the second segmentation may be performed according to the method 200 shown and described with respect to FIG. 4, except that the refinement zone 64 using method 200 is a refinement zone determined using network 370-1. In such implementations, the use of network 30-1 and the determination of the coarse estimate for the coordinates of the outer surface of the targeted feature, tumor 44, may be omitted.

FIG. 5 further illustrates a second alternative user selectable mode in which the physician or other healthcare worker may use an input 380 to select the inner boundary 66 and/or the outer boundary 68 of refinement zone 64. The input 380 may be in the form of a mouse and displayed cursor by which the physician or healthcare worker may draw the boundaries of refinement zone 64. The input 380 may be in the form of a touchscreen and a stylus by which the physician or healthcare worker may draw the boundaries of the refinement zone 64. In some implementations, system 320 may present on the display various user selectable refinement zones from which the physician or healthcare worker may select or move for use in performing the segmentation that is used to determine the coordinates for outer surface 72. Such input may be given in three dimensions or in one or more planes.

In some implementations, system 320 prompts the physician or healthcare to enter or select the size, shape and location of refinement zone 64 in the real-time ultrasound image after the coarse estimate for the outer surface coordinates of the targeted feature have been determined using model 30-1 (as described above) and while the coarse estimate is being displayed. In some implementations, system 320 may present multiple selectable refinement zones having different sizes, shapes and locations, wherein each refinement zone may offer a different degree of reliability for capturing the actual edge or outer surface of the targeted feature and may also have a different estimated processing time, based upon the previously determined coarse estimations for the outer surface of the targeted feature and the size of the particular refinement zone. The physician or healthcare worker may select one of the available refinement zones.

In some implementations, system 320 may omit the above-described coarse segmentation, wherein the physician or healthcare worker inputs or selects a refinement zone 64 on the displayed real-time ultrasound image without the coarse segmentation of the targeted feature. Once the refinement zone has been entered by the physician or healthcare worker, the refined higher resolution segmentation as described above may be performed by processor 24 using the input or selected refinement zone 64.

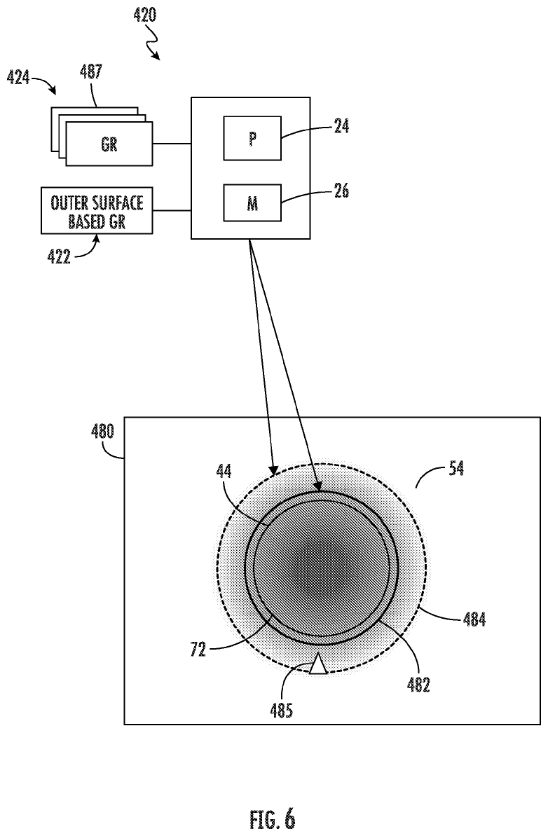

FIG. 6 is a diagram illustrating portions of an example machine learning system 420 for real-time segmentation of clinically relevant features during a surgical procedure. FIG. 6 illustrates an example of how inner and outer surgical guard rails may be determined and, in some implementations, visually presented, for guiding the movement of a surgical tool, such as cutting tool, along a surgical path, such as a cutting path, proximate to the segmented outer surface of the targeted feature. System 420 may be similar to system 20 or system 320, including all of their above describes features and functions, except that system 420 additionally determines an inner surgical guidance guard rail 482 and an outer surgical guidance guard rail 484. Those components of system 420 which correspond to components of system 20 are system 320 are numbered similarly and/or are shown and described with respect to FIGS. 1-5.

The guard rails 482 and 484 serve as boundaries for guiding the path or trajectory of a surgical tool, such as a cutting tool 485. With respect to the removal of a tumor, the inner guard rail 482 defines an inner most boundary for the cutting path that provides a satisfactory degree of confidence that the entire tumor 44 will be cut away and removed. In other words, if the cutting tool path intercepts the inner boundary 482 and moves inward of the inner boundary, there is a greater chance that the entirety of the tumor may not be removed. The outer guard rail 484 defines an outer most boundary for the cutting path that attempts to ensure that the entire tumor 44 will be cut away while also reducing or minimizing the removal of otherwise healthy features or tissue. In other words, if the cutting tool path intercepts the outer boundary and moves outward of the outer boundary, otherwise healthy tissue may be unnecessarily cut or removed.

In the example illustrated, system 420 offers two user selectable modes for determining guard rails 482, 484. In a first mode 422, system 420 determines at least one of the inner guard rail 482 and the outer guard rail 484 using the segmented outer surface 72 of the targeted feature, the tumor 44. When operating in such a mode, the inner guard rail 482 and the outer guard rail 484 may be based upon the second higher resolution feature edge segmentation determined using network 30-2 as described above. In some implementations, the inner guard rail 482 and the outer guard rail 484 may be based upon the above-described coarse or rough segmentation determined by network 30-1 as described above, wherein the second finer segmentation may or may not be performed.

In some implementations, the inner guard rail 482 may coincide with the estimated coordinates for the outer surface based upon the coarse segmentation using network 30-1 or may coincide with the estimated coordinates for the outer surface based upon the refined, second segmentation using network 30-2. In other words, the segmented outer surface serves as the inner guard rail 482. In some implementations, the first, coarse feature edge segmentation or the second finer feature edge segmentation may be inflated by a first amount to define the inner guard rail 482. In some implementations, the first coarse feature segmentation or the second finer feature edge segmentation may be inflated by a second greater amount to define the outer guard rail 484. Inflation of a feature edge segmentation refers to the uniform outward movement or spacing of the feature edge segmentation along the perimeter of the feature edge segmentation. In some implementations, the size, shape and location for one of the guard rails 482, 484 may be automatically determined based upon either the segmentation resulting from model 30-1 or the segmentation resulting from model 30-2, whereas the other of the guard rails 482, 484 is directly input by a physician or healthcare worker such as with input 380.

In a second mode 424, system 420 automatically determines one or both of guard rails 482, 484 using a network. When operating in such a mode, system 420 utilizes a trained processor that is been trained using a set of training ultrasound images 487, wherein each of the images 487 depicts a targeted feature, such as tumor 44, and training versions of one or both of guard rails 482, 484. Such training ultrasound images 487 may be in the form of a historical ultrasound image, a synthetic ultrasound image, a versioned ultrasound image, or combinations thereof. Using such training images 487, the network is trained to determine and present the inner guard rail 482 and/or the outer guard rail 484 in a real-time ultrasound image presented on display 480. The physician or healthcare worker, or an automated robotic system, may utilize the guard rails 482, 484 for determining, planning or controlling movement of a surgical tool, such as cutting tool 485, wherein the surgical path is to be kept within or between guard rail 482, 484.

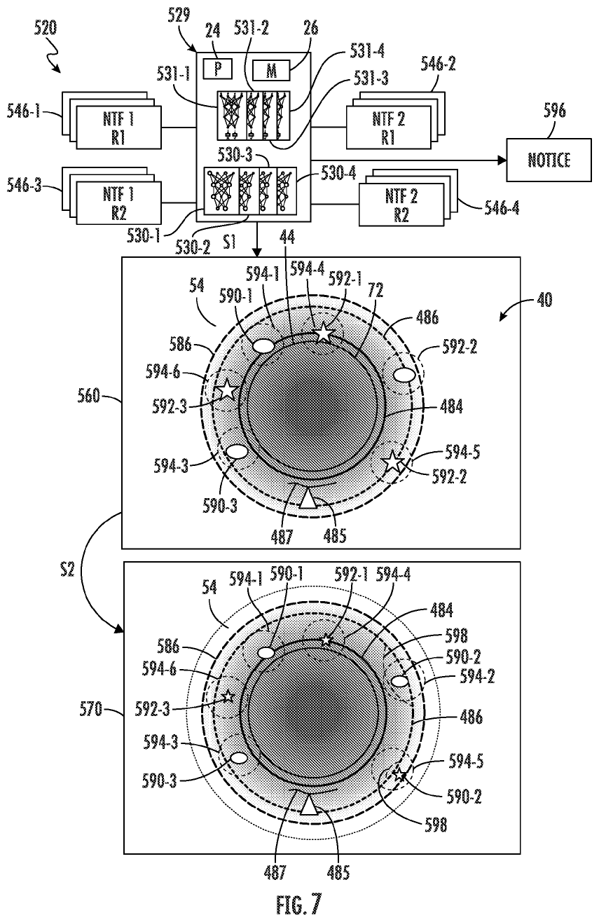

FIG. 7 is a diagram schematically illustrating portions of an example machine learning system 520 for real-time segmentation of clinically relevant features during a surgical procedure. FIG. 7 illustrates an example of how the machine learning systems 20, 320 and/or 420 may additionally segment non-targeted features proximate to a targeted feature in a real-time ultrasound image. System 520 may be similar to system 20, system 320 and/or system 420 described above, including all of their components and functions, except that system 420 additionally segments non-targeted features, such as arteries, veins and nerve bundles, which may be proximate to the targeted feature. Those components of system 520 which correspond to components of system 20, system 320, or system 420 are numbered similarly and/or are shown and described with respect to FIGS. 1-6.

Memory 26 comprises a non-transitory computerized readable medium containing instructions for directing processor 24 to determine the shape, size, and coordinates for a segmentation buffer 586 in the real-time ultrasound image 40, an enlarged portion of which is presented on display 560. Segmentation buffer zone 586 constitutes a region proximate to the segmented outer surface 72 where system 520 is to segment non-targeted features that may impact the timing, safety or effectiveness of the surgical operation to be performed using the cutting tool 485. Examples of non-targeted features may include, but are not limited to, blood vessels (arteries/veins) and nerve bundles.

In some implementations, the segmentation buffer zone 586 is based upon the anticipated cutting path of the cutting tool 485. In some implementations, the segmentation buffer zone 586 is based upon the previously determined inner guard rail 482 and the previously determined outer guard rail 484. For example, in some implementations, the segmentation buffer zone 586 has inner and outer boundaries that coincide with the inner guard rail 482 and the outer guard rail 484, respectively. In some implementations, the inner boundary of the segmentation buffer zone may be slightly inward of the inner guard rail 482 so as to segment any non-targeted feature that may be located between the inner guard rail 482 and the segmented outer surface 72 of the tumor 44. In some implementations, as indicated by broken lines, the outer boundary of the segmentation buffer zone may extend outwardly of the outer guard rail 484 such that non-targeted features slightly outside or adjacent to the outer guard rail 484 will also be segmented. Because segmentation buffer zone 586 defines a smaller portion of the larger real-time ultrasound image 40 for segmenting non-targeted features, processing timer bandwidth is reduced.

In the example illustrated, system 520 carries out segmentation of non-targeted features using a network 529 which may be a single network or which may comprise subnetworks such as the example subnetworks 530-1, 530-2, 530-3, 530-4 (collectively referred to as subnetworks 530) and the example subnetworks 531-1, 531-2, 531-3 and 531-1 (collectively referred to as subnetworks 531). In some implementations, subnetworks 530 may be a first network while subnetworks 531 are part of a second network. In the example, system 520 performs a first coarse segmentation of non-targeted features using down sampled data from the real-time ultrasound image at a first resolution followed by second finer segmentation, at a second resolution greater than the first resolution, using up sampled data contained within a smaller region of the real-time ultrasound image, wherein the size, shape and size or location of the smaller region is based upon the first coarse segmentation. As shown by display 560, processor 24 performs the first coarse segmentation of non-targeted features in the real-time ultrasound image 40 using subnetworks 530-1 and 530-2 which have been trained to segment different types of non-targeted features in a real-time ultrasound image at a first resolution. In other embodiments one network can detect the targeted and non-targeted features (e.g. 530 (coarse) and 531 (fine)). In the example illustrated, networks 530-1 and 530-2 have been trained to segment blood vessels and nerve bundles, respectively. Subnetwork 530-1 comprises a machine trained model or network created during a training phase or mode of an in-training subnetwork 531-1 using or based upon a set of ultrasound images 546-1 having a first coarse or lower resolution R1. The model or subnetwork 531-1 performs multiple iterations with the training data set of ultrasound images 546-1 to learn those particular features or characteristics corresponding to the particular non-targeted feature, such as a blood vessels.

Similarly, subnetwork 530-2 comprises a machine learning model or network created during a training phase or mode of an in-training subnetwork 531-2 using or based upon a set of ultrasound images 546-2 having the first coarse or lower resolution R1 and depicting a particular non-targeted feature. The model or subnetwork 531-2 performs multiple iterations with the training data set of ultrasound images 546-2 to learn those particular features or characteristics corresponding to a second particular non-targeted feature, such as a nerve bundles.

System 520 carries out the coarse segmentations for the blood vessels and nerve bundles by down sampling data from the real-time ultrasound image, the down sampling corresponding to the coarse resolution of the training ultrasound images 546-1 and 546-2. In the example illustrated on display 560, system 520 has identified and segmented blood vessels 590-1, 590-2, and 590-3 in the segmentation buffer zone in the real-time ultrasound image 40 using the machine trained subnetwork 530-1. System 520 has identified and segmented nerve bundles 592-1, 592-2 and 592-3 within the segmentation buffer zone 586 using the machine learning subnetwork 530-2. In some implementations, the segmentation of non-targeted features may end following such a segmentation.

However, in the example illustrated, processor 24, following instructions contained in memory 26, proceeds by determining smaller refined segmentation zones 594-1, 594-2, 594-3, 594-4, 594-6 and 594-6 (collectively referred to as zones 594), based upon the prior coarse segmentation of blood vessels 590-1, 590-2, 590-3, nerve bundles 592-1, 592-2 and 592-3, respectively. The segmentation of the real-time ultrasound image 40 at a higher resolution more precisely determines the coordinates of the blood vessels 590 and the nerve bundles 592. Because the second finer segmentation is restricted to the smaller segmentation zones 594, less data is processed, facilitating output of the second segmentation of the nontargeted features in less time using less computing power or bandwidth.

As shown by display 570, processor 24, following instructions contained in memory 26, utilizes the refinement zones 594 to select those regions or portions of ultrasound image 40 for performing a second volumetric segmentation (S2) of non-targeted features, blood vessels 590 and nerve bundles 592, at a higher resolution to estimate a more precise configuration of each of the non-target features. To perform the second segmentation at the high-resolution, processor 24 applies the machine learning model or subnetwork 530-3 to the up sampled data from the live or real-time ultrasound image 40 within each refinement zone 594. The data corresponding to the real-time ultrasound image 40 within each zone 594 is sampled at a rate to match or closely approximate the resolution R2 of the training set of ultrasound images 546-3 and 546-4 used to train subnetworks 530-3 and 530-4, respectively. Processor 24 uses subnetwork 530-3 to perform the second refined segmentation of the up sampled volumetric data from image 40 to infer or estimate the more precise locations of blood vessels 590. Likewise, processor 24 uses subnetwork 530-4 to perform the second refined segmentation of the upper sampled volumetric data from image 40 to infer or estimate the more precise locations of nerve bundles 592. Subnetworks 530-3 and 530-4 are created during a training phase or mode of in training subnetworks 531-3, 531-4 using or based upon set of ultrasound images 546-3 and 546-4, respectively. Each of the training images 546-3 and 546-4 have a second resolution R2 greater than the first resolution R1 and depict a particular non-targeted feature. The model or subnetwork 531-3 performs multiple iterations with the training data set of ultrasound images 546-3 to learn those particular features or characteristics corresponding to a second particular non-targeted feature, such as a blood vessel. The model or subnetwork 531-4 performs multiple iterations with the training data set of ultrasound images 546-4 to learn those particular features or characteristics corresponding to a second particular non-targeted feature, such as a nerve bundle.

The higher resolution sampling of data from image 40 (relative to down sampling of the data for the first coarse segmentation) and the use of the training set of ultrasound images 546-3, 546-4 at the resolution R2 increases the precision of the size and location of the segmented non-target features. At the same time, because the higher resolution segmentation which analyzes a greater percentage or number of pixels is limited to those regions within each of the smaller segmentation zones 594, the overall number of pixels that are processed and input into subnetworks 530-3 and 530-4 is reduced, reducing the processing time for performing this second or refined segmentation of the outer surface.

As shown by display 570, the segmentation of the non-targeted features may result in particular non-targeted features moving into or moving out of the region between the guard rails which may impact the planned cutting path 47 of the cutting tool 45. In response to segmented non-targeted feature having coordinates within or between guard rails 482 and 484, processor 24 may automatically output a notice 596 to a physician or healthcare worker, wherein the notice indicates the presence of the particular blood vessel 590 or the particular nerve bundle 592 between the guard rails.

In some implementations, the system 520 may perform a multi-class segmentation of the segmented non-targeted features. For example, the system 520 may classify or identify the non-targeted features as a blood vessel or nerve bundle. In some implementations, the identification may be carried out by a network trained to identify different classifications or types of non-targeted features in an ultrasound image. In some implementations, the warning or notification 596 may be varied depending upon the classification/type of the non-targeted feature, its size, its location relative to the inner guard rail, its location relative to the outer guard rail, and/or a loss in organ or patient anatomical functioning should the non-targeted feature be severed or damaged.

In some implementations, system 520 performs both segmentation and classification of the non-targeted features such as nerve bundles and blood vessels (concurrently or automatically with one another). The classification may identify the presence of the non-targeted feature and may additionally identify the type of the non-targeted feature. The segmentation may identify the particular boundaries, size and/or coordinates of the non-targeted feature. In some implementations, system 520 may initially determine the presence of a non-targeted feature, whether the non-targeted feature is present in the ultrasound image, before proceeding with segmentation to identify the location, size or particular boundaries of the non-targeted feature. A region of an ultrasound image may not be segmented if system 520 determines that the region does not contain any non-targeted features or in response to the region not containing a particular type of non-targeted feature. In response to determining the presence of a non-targeted feature or the presence of a predetermined specific type of non-targeted feature, system 520 may proceed with segmenting a region of the ultrasound image. In such an implementation, computing bandwidth in time may be preserved in those situations where a region does not contain any non-targeted features or does not contain particular types of non-targeted features that may warrant further segmentation.

As indicated by broken lines on display 570, in some implementations, in lieu of the notice 596 or in addition to the notice 596, system 520 may automatically adjust the shape, spacings or location of the guard rails 484, 486 based upon the identified presence of a non-targeted feature between the guard rails 484, 486 or nearby the guard rails 484, 486. In the example illustrated, the outer guard rail 484 is adjusted, bent inwardly, such that the segmented blood vessel 590-2 and the segmented nerve bundle 592-2 are no longer between the now modified guard rails 484, 486. For example, processor 24 may automatically inwardly move the coordinates of those portions 598 of the outer guard rail 484 to establish a predetermined safety clearance with respect to blood vessel 590-2 or nerve bundle 592-2.

In some implementations, system 520 provides the physician or other healthcare worker with the opportunity to manually adjust the shape or location of guard rail 482 and/or guard rail 484 based upon the segmented non-targeted features presented on display 570. For example, display 570 may comprise a touchscreen, wherein the physician may use a stylus to draw a new modification. In some implementations, system 520 may have an input including a mouse and a depicted cursor which is moved to draw the revised shape of guard rail 482 or guard rail 484. In some implementations, system 520 may present multiple user selectable revisions to guard rail 482 and/or guard rail 484.

Targeted features may be contained entirely within an organ and spaced from an outer surface of the organ. Such targeted features will be referred to herein as endophytic targeted features. In contrast, other tumors may be located such that a portion of the tumor is on the surface of the organ and part of the tumor is contained within the organ itself. Such tumors will be referred to herein as exophytic targeted features. In order to excise an endophytic targeted feature, such as a tumor, from the organ it is necessary to cut through the outer surface of the organ to reach the tumor itself. A proposed cutting path from the outer surface of the organ to the tumor may pass through non-targeted features that would impair the function of the organ.

In one implementation, system 520 receives, via input 380, a proposed cutting path from the surface of the organ to the endophytic tumor and conducts a segmentation of a region having a predetermined volume about the cutting path to identify any non-targeted features of interest. The process of segmentation of the predetermined volume of the proposed cutting path is similar to the segmentation of the buffer zone as described herein.

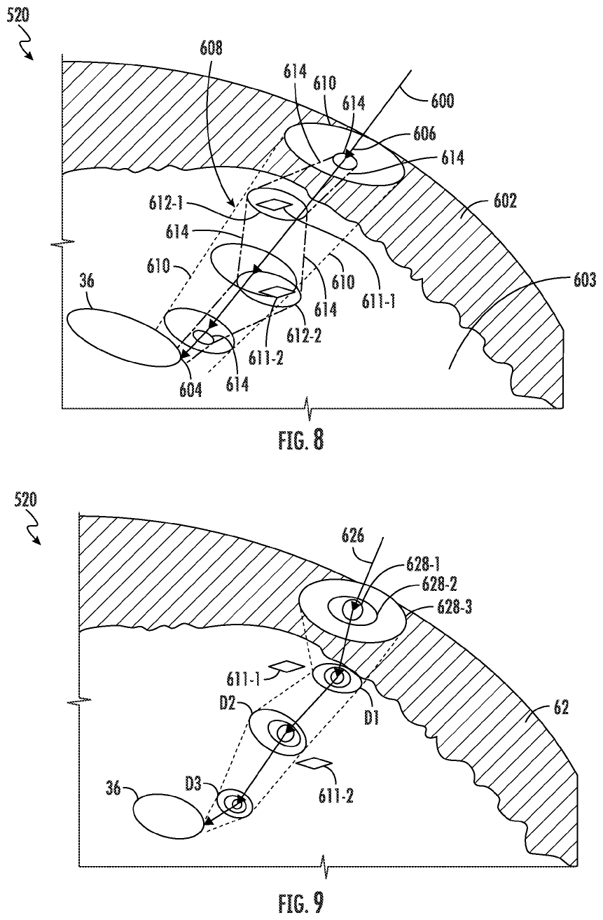

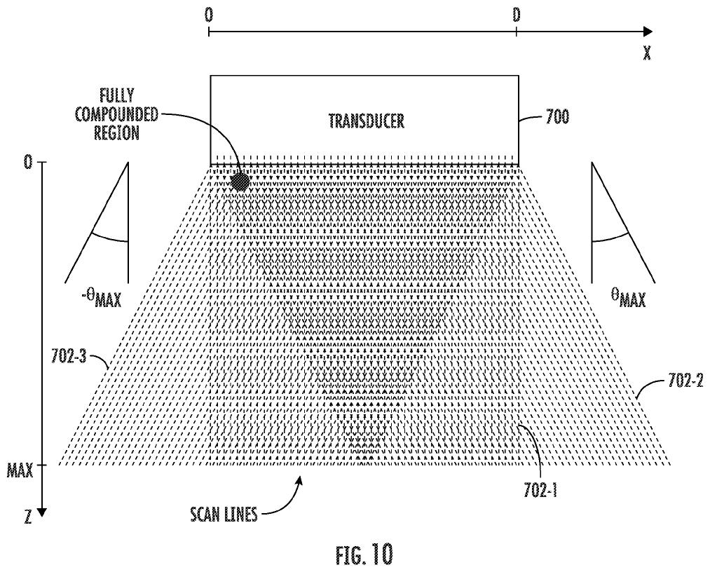

FIGS. 8 and 9 are diagrams schematically illustrating system 520 determining an example cutting path to an endophytic tumor. FIG. 8 illustrates an example multistep or multistage segmentation along and about an initial cutting path. In the example illustrated, system 520 (instructions in memory 26 directing processor 24) may begin with a substantially straight-line cutting path 600 which is either input by a person or automatically determined by system 520 based upon the previously identified edges of the feature of interest, such as the edges of a tumor 36. As shown by FIG. 8, cutting path 600 passes through the surface 602 of the organ and proceeds through an interior portion 603 of the organ until reaching a predetermined edge or a cutting start location 604. The initial entry point 606 for cutting path 600 and the trajectory of cutting path 600 may be calculated or determined so as to avoid any organ surface ultrasound probe, which may lie on an opposite side of the same organ, and so as to shorten the length of the path 600 to start location 604. Reducing the length of path 600 reduces the amount of healthy tissue of the organ that must be cut or otherwise be disturbed.

Once provided with the initial entry point 606 and the initial cutting path 600, system 520 carries one or more segmentation routines to refine and alter the cutting path. In the example illustrated, system 520 performs a coarse or rough segmentation in a cutting path segmentation zone 608 (outlined by broken line 610). Cutting path segmentation zone 608 comprises regions or volumes about the initial cutting path 600, extending from surface 602 to the tumor 36. In some implementations, the cutting path segmentation zone 608 is automatically determined based upon the previously determined initial cutting path 600. For example, the cutting path segmentation zone may constitute a tubular volume centered about and containing the initial cutting path 600. In some implementations, the cutting path segmentation zone 608 may comprise a volume i. In some implementations, the volume may comprise a cone. In some implementations, the volume may have an oval cross-sectional shape. In some implementations, radius of the tubular volume may vary along the length or trajectory of the initial cutting path 600. In some implementations, the radius or width of the tubular volume may vary along the length of the initial cutting path 600 as a function of a distance from surface 602 and/or a distance from the cutting start location 604.

In some implementations, the shape of the cutting path segmentation zone may be based on default parameters such as a predefined radius of the tubular volume from the initial cutting path 600. In some implementations, system 520 may prompt for input from a healthcare provider or other person, the input designating the predefined radius or what particular function should be used to define the cutting path segmentation zone 608. In some implementations system 520 may display the initial cutting path 600 and may receive input from a healthcare provider or other person indicating the boundaries or shape of the cutting path segmentation zone 608. For example, a healthcare provider may utilize a touchscreen, a stylus, a mouse or other tool to directly point to or draw the boundaries of the cutting path segmentation zone 608 on the display depicting the initial cutting path 600.

The initial coarse segmentation carried out in the cutting path segmentation zone 608 may roughly or coarsely identify non-targeted features (arteries, veins, nerve bundles or the like) that may lie on or near the initial cutting path 600. The resolution of the segmentation in the cutting path segmentation zone 608 may be the same as the resolution of the segmentation used to determine the refinement zone for the surface of the targeted feature or tumor, may be the same as the resolution of the segmentation used in the refinement zone, or may be any other level of resolution. This first coarse segmentation about the initial cutting line 600 may be performed using a first machine learning model or network that is been trained based upon ultrasound images at a first resolution depicting the non-targeted feature of interest. This segmentation may be carried out on a down sampled set of data from the cutting path segmentation zone in the real-time ultrasound image. In the example illustrated, the rough segmentation within cutting path segmentation zone 608 identified nontargeted features 611-1 (vascular feature) and 611-2 (a nerve bundle).

Thereafter, the coarse segmented non-targeted feature coordinates may be used as a basis for defining a smaller non-targeted feature refinement zone about the non-targeted feature. Similar to the refinement zone used to refine the estimated location of the edge of the targeted feature, such as the edge or outer surface of the tumor, the smaller non-targeted feature refinement zone may be used to refine the coordinates, size, orientation or the like of the non-targeted feature.

In the example illustrated, system 520 has identified nontargeted feature refinement zone 612-1 and 612-2 (collectively referred to as refinement zones 612) based upon the coarsely identified nontargeted features 611-1 and 611-2, respectively. In some implementations, the non-targeted feature refinement zones may have a single outer boundary completely surrounding the coarse estimate for the boundary of the non-targeted feature (a sphere or other three-dimensional shape). In some implementations, the non-targeted feature refinement zones may be a ring or annular in shape (e.g., a volumetric or three-dimensional donut), having an inner boundary and an outer boundary, wherein the coarsely identified edges or perimeter of the non-targeted feature lies within the circular, oval or amorphous shaped ring.

In some implementations, system 520 (its processor and associated non-transitory computer readable medium containing instructions for the processor) automatically determines the boundaries of the non-target refinement zone 612 about the roughly determined non-targeted feature 611. System 520 may inflate the roughly determined non-targeted feature boundary to define and outer volumetric boundary of the refinement zone may deflate the roughly determined non-targeted feature boundary to define an inner volumetric boundary of the non-targeted feature refinement zone. In some implementations, system 520 may automatically define the outer boundary of the non-targeted feature refinement zone based upon a predetermined distance from a coarsely determine outer edge or a coarsely determined center point of the non-targeted feature. In some implementations, system 520 may display the coarse or roughly estimated position of the non-targeted feature, wherein a healthcare provider other person may manually input the outer boundary or the outer boundary and inner boundary of the non-targeted feature with a stylus, mouse, touchscreen and the like.

Once the non-targeted feature refinement zones 612 have been determined or identified, a second machine trained model or network, trained based upon ultrasound images at a second resolution, greater than the first resolution, and depicting the non-targeted feature of interest may be used. The second machine trained model or network samples voxels within the zones 612 of the real-time ultrasound image at the second resolution when carrying out the higher resolution segmentation of the non-targeted feature. The second machine trained model or network may more precisely define the edges of the non-targeted feature to more precisely define its location. Because the second non-targeted feature segmentation is carried out using a second network trained at a higher resolution and employing higher resolution of data sampling in the non-targeted feature refinement zones of the real-time ultrasound image (analyzing a greater number of voxels per area or volume), the resolution of the estimated edge of each of the non-targeted features is enhanced. At the same time, because the second non-target feature segmentation is applied to just the non-targeted feature refinement zone, the total number of voxels being analyzed is reduced (as compared to a circumstance where every voxel within the cutting path segmentation zone were sampled or used), reducing computational time and permitting the overall segmentation of the feature to be more likely performed in real-time or with less processing resources. Because such coarse and fine segmentation of the non-targeted features may be carried out in real-time, such segmentation will reflect changes in the coordinates or locations of the edges of the non-targeted features that may result due to deformation of the organ or tissue as the cutting tool/effector engages the organ.

As indicated by broken line 614, in some implementations, system 520 may additionally perform the segmentation at the second higher resolution on additional volume within or near the original volume of the refinement zone 608. In the example illustrated, system 520 carries out the second higher resolution segmentation in those volumes extending between the non-targeted feature refinement zones 612, the volume extending between the non-target refinement zone 612-1 and the entry point 606, and the volume extending between the non-target refinement zone 612-2 and the cutting start location 604. In the example illustrated, these additional volumes taper in directions away from the non-targeted feature refinement zones 612. With such tapering, those volumes nearer to the non-targeted feature refinement zones 612 are larger as compared to those portions of the volumes further away from the previous identified non-targeted feature. Such tapering conserves computing bandwidth while at the same time maintaining the ability to identify other non-targeted features, (potentially smaller in size and not detected by the coarse segmentation) that are located near the coarsely identified non-targeted features 611.

System 520 uses the identified coordinates of the non-targeted features 611 that may lie on or nearby the initial cutting path 600 to modify the initial cutting path 600. In some implementations, system 520 displays the initial cutting path and the identified locations of the non-targeted features while providing a healthcare provider or other person the opportunity to input modifications to the initial cutting tool path based upon the locations of the non-targeted features. For example, healthcare providers may utilize a stylus, mouse or touchscreen to manually draw the revised cutting path or to move or change shape of particular segments of the initial cutting path so as to avoid the identified non-targeted features. This modified cutting tool path may be stored and subsequently utilized to guide movement of a cutting tool or effector to the initial cutting starting point 604. Such movement of the effector may be automated in a robotic fashion or may be manually performed by a healthcare provider, wherein the stored and modified cutting tool path is used to guide the healthcare provider when controlling movement of the effector or cutting tool.

In some implementations, the system 520 may classify the segmented non-targeted features. For example, the system 520 may classify the non-targeted features as a blood vessel or nerve bundle. In some implementations, the classification may be carried out by a machine learning network trained to identify different classifications of non-targeted features in an ultrasound image. In some implementations, system 520 may output a warning or notification as a cutting tool is moving along the cutting tool path, the warning being based upon the proximity of the cutting tool to the determined location of the non-targeted feature along or near the cutting tool path. Such a notice or warning may be varied depending upon the classification of the non-targeted feature, its size, and/or a loss in organ or patient anatomical functioning should the non-targeted feature be severed or damaged.

In some implementations, system 520 may determine multiple possible cutting tool paths 600. In some implementations, system 520 may display such available cutting tool paths 600 for selection by a healthcare provider. In some implementations, system 520 may carry out such multi-stage segmentation for each of any multiple possible initial cutting tool paths 600. In such implementations, system 520 may present multiple resultant parameters or characteristics for each of the available cutting tool path 600, providing information for the selection of which available cutting tool path 600 is to serve as the basis for a modified cutting tool path. For example, system 520 may present information such as how much tissue is cut or disturbed by each of the possible cutting tool path, onto organ functionality is affected by each of the possible cutting tool path, how precise the cutting tool must be for each of the cutting tool paths, how much time is required for each of the cutting tool paths, and the like.

In some implementations, the corners or locations of the non-targeted features along a cutting tool path may be determined in other manners. For example, the coordinates of non-targeted features may be determined using stereoscopic data, preoperative CT data or combinations thereof.

In some implementations, system 520 may determine, and potentially display a cutting tool path guide. FIG. 9 is a diagram schematically illustrating an example cutting tool path guide determined by system 520 based upon the determined locations of the non-targeted features 611, the entry point 606 and the cutting starting point 604. FIG. 9 illustrates an example where the aforementioned cutting tool path 600 has been modified, resulting in the revised cutting tool path 626. Based upon the revised cutting tool path 626, system 520 may generate, and potentially display, various path guides 628-1, 628-2 and 620 (collectively referred to as path guides 628). Path guides 628 are volumetric or 3D in nature, forming a tubular shape structure that extends about the modified cutting tool path 626 and further extends from surface 602 to the starting cutting point 604. In the example illustrated, path guides 628 are illustrated on the surface 602 and at three distinct subsurface slices that pass through the path guides. Prior to the cutting tool entering the entry point 606. The path guides on the surface 602 may be displayed. As a cutting tool passes through surface 602 and moves below surface 602, the displayed portion or slice of path guides 628 may change. For example, when the effector or cutting tool has reached the depth D1, the path guides 628 at depth D1 will be displayed. When the cutting tool or effector has reached depth D2, the path guides 628 at depth D2 will be displayed. When the cutting tool has moved to depth D3, those portions of the path guides 628 at depth D3 will be displayed. Such path guides may change in size and/or shape at each of the different depths. Such path guides may be in the form of a cylinder, a cone, an amorphous tubular shape or the like. Such path guides may taper, widen or change shape depending upon a proximity of any nontargeted feature, the importance of the non-targeted feature and other factors. Such path guides provide various cutting tool path tolerance regions to guide actual movement of the cutting tool along the modified cutting tool path 626. For example, movement of the cutting tool during the surgical procedure may vary from the recommended cutting tool path 626, intersecting different path guides.

Each individual path guide 628 represents a recommendation level or confidence level for the actual cutting tool path being taken. In the size and shape of the collective group of guides 628 indicates an amount of variability that is allowable as the cutting tool is moved through the tissue of the organ. For example, in some implementations, the collective group of guys guides 628 may have a smaller size of the different shape and gaps corresponding to depths where a non-targeted feature has been located.

In the example illustrated, path guides 628-1 represents the highest recommended region for the intersection point at the associated depth for the cutting tool path. Path guides 628-2 represents an intermediate confidence or intermediate recommended location for the intersection of the cutting tool path at the associated depth. Path guides 628-3 represent the lowest acceptable range or region of intersection points for the cutting tool path at the associated depth. In some implementations, as a practitioner moves the cutting tool path through the various depths, different visual or audible warnings or notices may be provided to the medical practitioner depending upon which of the three bands is currently being intersected by the current path of the cutting tool. In particular implementations, the processor of the example artificial intelligence or machine learning system may further restrict movement of a surgical tool, such as a cutting tool, along its path within the particular path guide 628.

In some implementations, guide 628 may not be displayed, but may be used as guiding thresholds for robotic system. In some implementations, the speed, cutting rate or other parameters associated with movement or operation of the cutting tool may be automatically adjusted depending upon which of the three path guides is currently being intersected by the cutting at the particular depth. Although system 520 is illustrated as utilizing three distinct path guides, in other implementations, a greater or fewer number of such path guides may be used and/or displayed. Although such path guides are illustrated as having oval shapes, the different path guides may alternatively have non-oval shapes, such as circular, polygonal, or amorphous cross-sectional shapes along such tubular guides.

Although guides 628 are illustrated for use and/or display prior to or during movement of the cutting tool or effector from an exterior of the organ to a tumor or other targeted feature within the organ, system 520 may likewise generate similar guides in a similar fashion for movement of the cutting tool or effector about a tumor or other targeted feature during their cutting and removal of the tumor or targeted feature. In such implementations, the cutting guides may comprise a tubular shape guide that, rather than extending from the surface of the organ to a tumor, extends about edges of the tumor. The individual slices of the guide, rather than being present at different depths, will occur at different angular positions about the tumor. For example, as a cutting tool moves around the perimeter of the tumor to exercise the tumor, a viewpoint looking forward from the cutting tool and including one or more guides similar to guide 628 may be determined and potentially displayed. As a cutting tool moves forward, different slices of the tubular guide extending around the tumor are presented to the surgeon or are used as a control guide for an automated robotic system. As discussed above, in some implementations, alerts, notifications are warnings may be output depending upon which of the guides is a being currently intersected or is about to be intersected by the cutting tool or effector.

In some implementations, system 520 provides the physician or other healthcare worker with the opportunity to manually adjust the shape or location of the path guides 628. For example, display 570 may comprise a touchscreen, wherein the physician may use a stylus to draw a new modification. In some implementations, system 520 may have an input including a mouse and a depicted cursor which is moved to draw the revised shape of path guides 628. In some implementations, system 520 may present multiple user selectable revisions to guard rail 482 and/or guard rail 484.

The network topology that has been described to segment volume ultrasound data consists of two networks, a coarse network and a fine network. The input to the network is 3D ultrasound data which may be rf, amplitude and phase (e.g., I/Q data), or even detected rf (amplitude only). One method to improve the image appearance/quality is by using spatial compounding. Spatial compounding looks at the same tissue region from multiple perspectives or imaging angles. FIG. 10 shows a region that can be detected through three different imaging angles. In the example illustrated, the transducer 700 is controlled to provide three imaging angles 702-1, 702-2 and 702-3. Imaging angle 702-1 is generally perpendicular to transducer 700. Imaging angle 702-2 is angled to the right (as seen in FIG. 10). Imaging angle 702-3 is angled to the left (as seen in FIG. 10.

In conventional diagnostic ultrasound, tissue is typically viewed from only one angle. This leads to an image where angle dependent scatterers may not be easily detected (e.g. vessel walls) or regions in the image with a mottled appearance which is referred to as speckle. Because compounding looks at the same region with multiple angles, the effect of angle-dependent targets are significantly reduced. Furthermore, the mottled appearance of speckle is minimized since the bright and dark regions tend to be averaged together. In the ideal case, the noise of the speckle region is reduced by the square root of the number of unique compound images. If volume ultrasound images are acquired, compounding may occur in three dimensions rather than just two as shown in the figure. It is hypothesized that having multiple observations of the same region as provided by compounding will lead to better feature extraction by the Al network. It is important to note that there are many methods to apply compounded data sets to segment a feature as well be described.

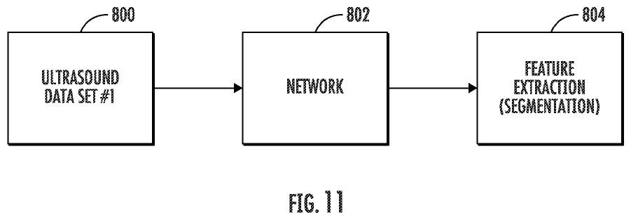

FIG. 11 shows the most basic system block description where one ultrasound data set 800 (one angle, one perspective) is analyzed by an Al, machine learning or other network 802 to perform segmentation 804 to extract features such as arteries, nerves, or a tumor.

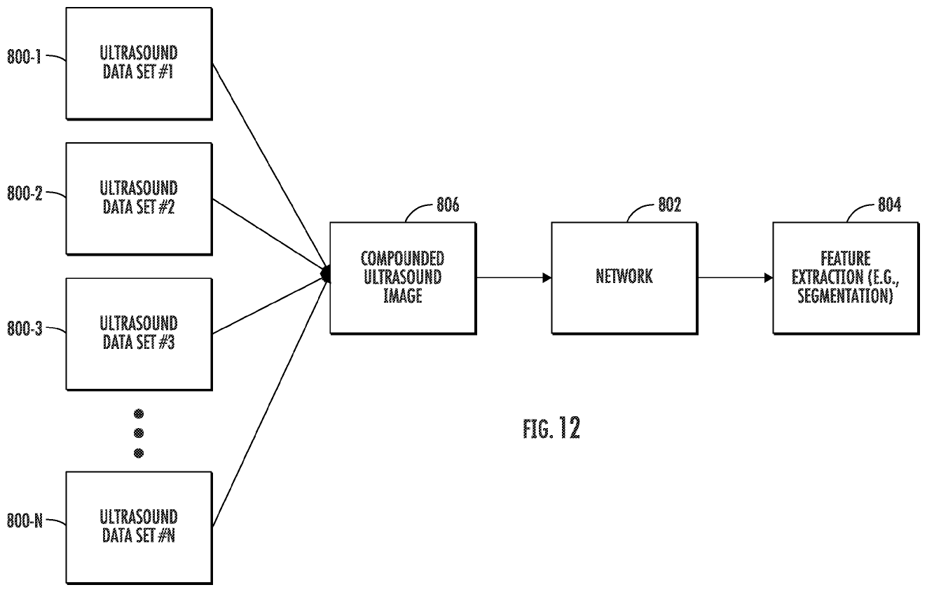

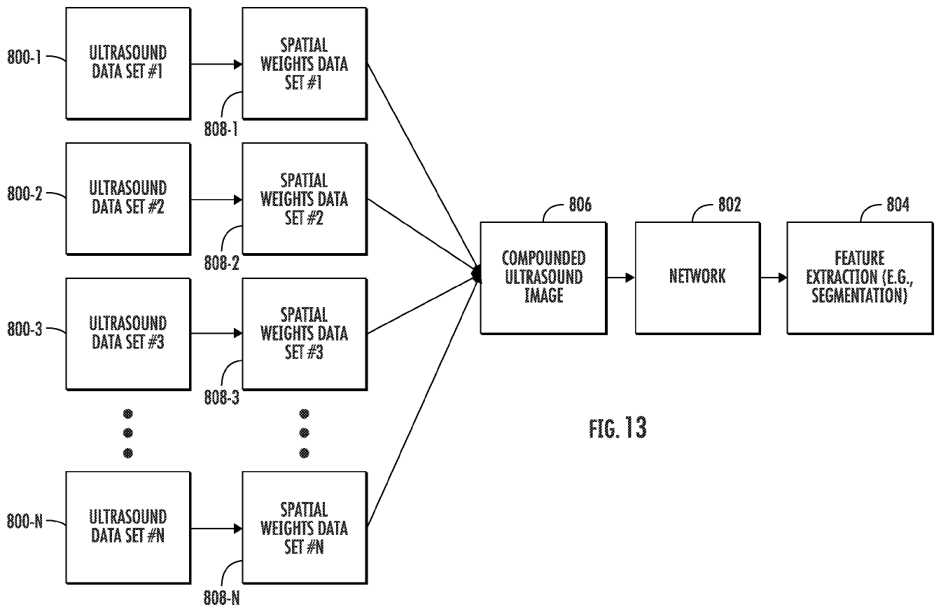

FIG. 12 shows a case where multiple ultrasound data sets 800-1, 800-2, 800-3 . . . 800-N (collectively referred to as data sets 800) are acquired of a similar region. These data sets 800 are combined to generate a compounded data set or image 806. The compounded data set 800 is then sent to an Al, machine learning or other network 802 where features 804 are extracted. It is hypothesized that because compounded imaging reduces speckle and improves the appearance of specular reflectors, that the detection of arteries, veins and tumors will be significantly enhanced.

FIG. 13 shows a similar case as FIG. 12 with the exception that spatial weights 808-1, 808-2, 808-3 . . . 808-N (collectively referred to as weights 808) are applied to the data sets 800-1, 800-2, 800-3 . . . 800-N, respectively. Although compounding reduces speckle and enhances specular reflectors, the images that are compounded together are typically just averaged. In other words, each image that interrogates the same region gets equally weighted together. This may not be the ideal approach to extract features in an ultrasound image using artificial intelligence. This is because in one perspective the ability to detect an edge may be dependent on the axial resolution whereas in another perspective the ability to detect the same edge may be dependent on the lateral resolution. Since the lateral resolution is typically worse than the axial resolution, an equally weighted approach may decrease the segmentation accuracy. Another approach is to apply weights to each point within the ultrasound data set. This method could be used to emphasize optimal imaging angles to a specular reflector. Similarly, this method could be used to emphasize optimal angles near the boundary of a tumor since axial resolution tends to be substantially better than lateral resolution in an ultrasound image. For example, if the propagation direction is parallel to the surface normal (axial resolution), then this should be weighted more than if the propagation direction is perpendicular to the surface normal (lateral resolution). These weighted images would then be combined to form a unique compounded image that is analyzed by the AI network which extracts the features. The weights for the same spatial point could still be summed together to equal 1.

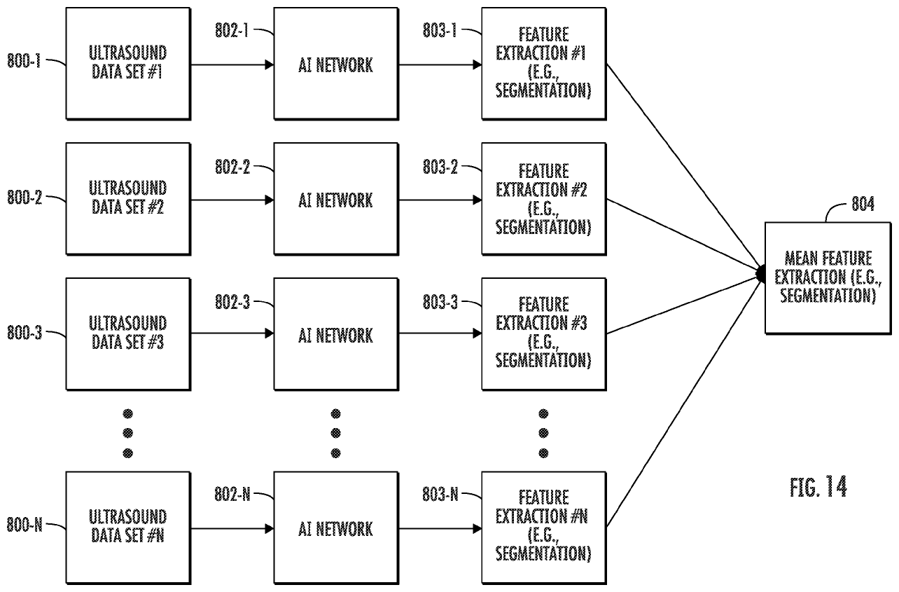

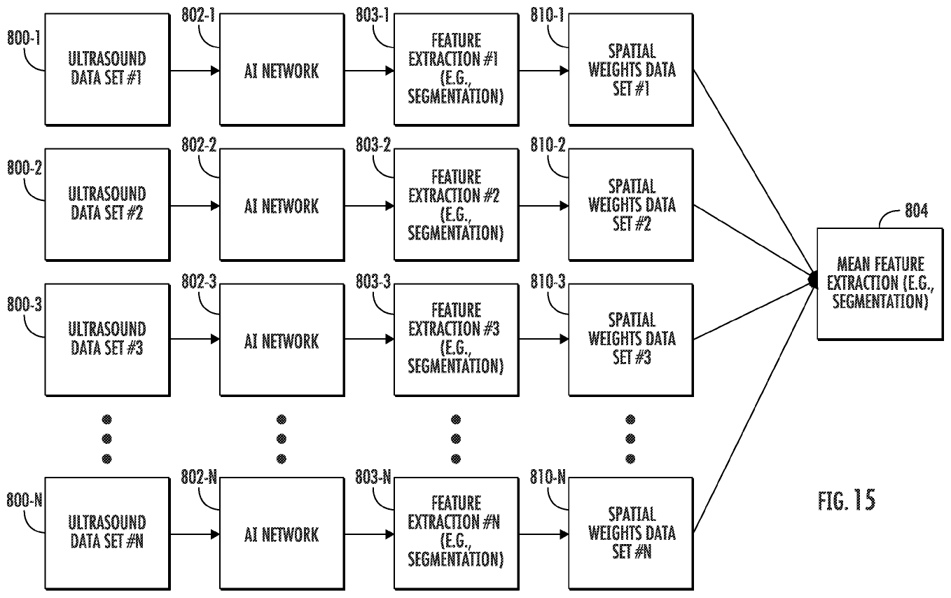

FIG. 14 treats each ultrasound data set 800-1, 800-2, 800-3 . . . 800-N independently as in FIG. 11 with a separate determination of the features 803-1, 803-2, 803-3 . . . 803-N (collectively referred to as features 803), respectively. After the features 803 are identified from each of data sets 800, the positions/boundaries of the segmented features are averaged together in the main feature extraction step 804 to produce a final result for each feature location/segmentation.

FIG. 15 shows a similar block diagram as FIG. 14 with the exception that spatial weights 810-1, 810-2, 810-3 . . . 810-N (collectively referred to as weights 810) have been placed on the extracted features 803-1, 803-2, 803-3 . . . 803-N, respectively. As described in FIG. 13, the weight assigned to the feature extraction may be dependent on the angle between the propagation direction to the surface normal as well as the relationship of the angle of the propagation direction to a specular surface if used in the feature extraction. The weighted results are averaged together in the main feature extraction step 804 to present the final extraction/location of the feature.

It is important to note that the weights applied in FIGS. 13 and 15 could be determined by other means other than the angle of the propagation direction to the surface normal or specular reflector. For example, the local signal-to-ratio of the signal could be used.

Although this description was for B-mode acquired data, it also applies to other ultrasound imaging modes and mixed modes. For example, ultrasound data set #1 could be B-mode data and ultrasound data #2 could be Doppler data.

Although the above disclosure largely focuses on the segmentation of targeted features (tumors) and non-targeted features in real-time ultrasound images, the above-described systems and methods may likewise be used to segment and identify the edges or coordinates of targeted features and nontargeted features in other imaging modalities. For example, the above-described systems and methods for segmenting the edges of a tumor or segmenting the edges of a non-targeted feature may be carried out on real-time optical images or other real-time imaging techniques or modalities.

Each of systems 20, 320, 420 and 520 may perform a segmentation of a clinically relevant feature in an ultrasound image during a surgical procedure in real time. Each of such systems 20, 320, 420, and 520 may present, in real-time, on a display, a depiction of the clinically relevant based on the segmentation. In the above examples, the systems carry out a multi-step segmentation process with a first coarse segmentation to define a refinement zone and a second fine segmentation within the refinement zone. In other implementations, each of such systems may perform a single stage step segmentation without the refinement zone or within the refinement zone defined in other fashions.

In other implementations, each of such systems may perform a multi-step segmentation process with more than two segmentations: a course segmentation and a fine segmentation. For example, a first segmentation of an ultrasound image at a first resolution may be utilized as a basis for determining boundaries of a first refinement zone. A second segmentation of the first refinement zone at a second resolution greater than the first resolution, may be utilized as a basis for determining boundaries of a second refinement zone at least partially within or overlapping the first refinement zone. A third segmentation of the second refinement zone at a third resolution greater than the second resolution may be utilized to determine an estimate for the boundaries of the clinically relevant feature or tumor. Alternatively, rather than the process may continue with additional segmentation that additional incrementally increasing resolutions until a satisfactory estimate or competence level for the boundary of the clinically relevant feature tumor is achieved. The above example processes may likewise be utilized when segmenting non-targeted features to identify the boundaries of such non-targeted features.

In some implementations, the example algorithms carry out real-time volume or three-dimensional segmentation of a targeted feature or clinically relevant feature. As should be appreciated, the example systems may likewise carry out two-dimensional segmentation of a targeted feature or clinically relevant feature. Such 3D or 2D segmentation may likewise be carried out on non-targeted features.

Although the claims of the present disclosure are generally directed to a machine learning system that performs a fine, higher resolution segmentation based on a prior course, lower resolution segmentation, the present disclosure is additionally directed to the features set forth in the following definitions.
Definition 1: A machine learning system for real-time segmentation of clinically relevant features during a surgical procedure, the system comprising:
   a trained processor to:
      perform a first segmentation of a real-time ultrasound image depicting a non-linear outer surface of a volumetric mass of a targeted feature based upon real-time ultrasound data at a first resolution;
      define a refinement zone based upon the first segmentation; and
      perform a second segmentation in the refinement zone of the real-time ultrasound image at a second resolution greater than the first resolution.
Definition 2: The machine learning system of Definition 1, wherein the refinement zone comprises an inner boundary and an outer boundary and wherein the outer surface of the targeted feature lies between the inner boundary and the outer boundary of the refinement zone.
Definition 3: The machine learning system of Definition 1, wherein the trained processor is to define an inner surgical guidance guard rail and an outer surgical guidance guard rail based upon the second segmentation, wherein a surgical tool path is to lie between the inner surgical guidance guard rail and the outer surgical guidance guard rail.
Definition 4: The machine learning system of Definition 3, wherein the trained processor is further configured output a recommended cutting path for a surgical tool, the cutting path being contained between the inner surgical guidance guard rail and the outer surgical guidance guard rail.
Definition 5: The machine learning system of Definition 3, wherein the trained processor is further configured to:
   define a segmentation buffer zone; and
   segment non-targeted features within the segmentation buffer zone.
Definition 6: The machine learning system of Definition 5, wherein the trained processor is further configured to adjust the outer surgical guidance guard rail based on segmented non-targeted features within the segmentation buffer zone.
Definition 7: The machine learning system of Definition 6, wherein the trained processor is further configured output a recommended cutting path for a surgical tool, the cutting path being contained between the inner surgical guidance guard rail and the outer surgical guidance guard rail.
Definition 8: The machine learning system of Definition 5, wherein the trained processor is further configured output a notice indicating regions between the inner surgical guidance guard rail and the outer surgical guidance guard rail where segmented non-targeted features are located.
Definition 9: The machine learning system of Definition 5, wherein the trained processor comprises:
   a first vision transformer to segment the non-linear outer surface of the volumetric mass of the targeted feature based upon real-time volumetric ultrasound data; and
   a second vision transformer to segment non-targeted features within the segmentation buffer zone.
Definition 10: The machine learning system of Definition 5, wherein the targeted feature comprises a tumor.
Definition 11: The machine learning system of Definition 5, wherein the segmentation buffer zone comprises an inner boundary and an outer boundary and wherein the refinement zone lies between the inner boundary and the outer boundary.
Definition 12: The machine learning system of Definition 5, wherein the segmentation buffer zone comprises an inner boundary and an outer boundary, the inner boundary coinciding with the refinement zone.
Definition 13: The machine learning system of Definition 5 further comprising an inner surgical guard rail and an outer surgical guard rail, wherein the inner surgical guard rail and the outer surgical guard rail lie between the refinement zone and an outer boundary of the segmentation buffer zone.
Definition 14: The machine learning system of Definition 5 further comprising an inner surgical guard rail and an outer surgical guard rail, wherein the outer surgical guard rail coincides with an outer boundary of the segmentation buffer zone.
Definition 16: The machine learning system of Definition 5 further comprising an inner surgical guard rail and an outer surgical guard rail, wherein the outer surgical guard rail is nonuniformly spaced from the inner surgical guard rail.
Definition 17: The machine learning system of Definition 5, wherein the outer surgical guard rail is shaped to exclude at least one of the segmented non-targeted features from between the inner surgical guard rail and the outer surgical guard rail.
Definition 18: The machine learning system of Definition 1, wherein the trained processor is configured to segment at least one of nerves and arteries within the segmentation buffer zone.
Definition 19: The machine learning system of Definition 1, wherein the trained processor is trained to segment the non-linear outer surface of the volumetric mass of the targeted feature based upon synthetic ultrasound data.
Definition 20: The machine learning system of Definition 1, wherein the trained processor is trained to segment features comprising at least one of nerves and arteries within the segmentation buffer zone based upon synthetic ultrasound data.
Definition 21: The machine learning system of Definition 1, wherein the segmentation buffer zone has a nonuniform width about the refinement zone.
Definition 22: The machine learning system of Definition 1, wherein the trained processor is configured to segment a first portion of the segmentation buffer zone at a first resolution and to segment a second portion of the segmentation buffer zone at a second resolution greater than the first resolution.
Definition 23: The machine learning system of Definition 1 further comprising a display, wherein the trained processor is configured to concurrently present boundaries of the segmentation buffer zone, with the segmented portion, and those features segmented in the segmentation buffer zone, on the display.
Definition 24: The machine learning system of Definition 1, wherein the trained processor is trained, based upon ultrasound images or synthetic ultrasound images to define the segmentation buffer zone.
Definition 25: The machine learning system of Definition 1, wherein the trained processor is to define a width of the segmentation buffer zone based upon the refinement zone.
Definition 26: The machine learning system of Definition 1, wherein the trained processor is trained to classify the targeted feature and to define a width of the segmentation buffer zone based upon the classification of the targeted feature.
Definition 27: The machine learning system of Definition 1, wherein the trained processor is trained to classify non-targeted features and to differently segment the non-targeted features based upon their classification.
Definition 28: The machine learning system of Definition 1, wherein the trained processor is configured to segment the non-linear outer surface of the volumetric mass of the targeted feature by successively applying different algorithms to smaller and smaller portions of the real-time volumetric ultrasound data, each of the different successive algorithms having a smaller down sampling of ultrasound data.
Definition 29: The machine learning system of Definition 1, wherein the trained processor is configured to:
   perform a third segmentation of a cutting tool path segmentation zone about a cutting tool path from a surface of an organ to the targeted feature within the organ based upon real-time ultrasound data at a third resolution;
   identify a non-targeted feature proximate the cutting tool path based on the third segmentation; and
   modify the cutting tool path based on the identified non-targeted feature.
Definition 30: The machine learning system of Definition 28, wherein the trained processor is configured to perform a fourth segmentation of a region containing the non-targeted feature proximate the cutting tool path, the fourth segmentation being based upon real-time ultrasound data at fourth resolution greater than the third resolution.
Definition 31: A machine learning system for real-time segmentation of clinically relevant features during a surgical procedure, the system comprising:
   a trained processor to:
      perform a first segmentation in refinement zone of a real-time image depicting a non-linear outer surface of a volumetric mass of a targeted feature based upon real-time image data at a first resolution;
      define a refinement zone based upon the first segmentation; and
      perform a second segmentation of the non-linear outer surface of the volumetric mass of a targeted feature based upon real-time image data within the refinement zone at a second resolution greater than the first resolution.
Definition 32: The machine learning system of Definition 31, wherein the refinement zone comprises an inner boundary and an outer boundary and wherein the outer surface of the targeted feature lies between the inner boundary and the outer boundary of the refinement zone.
Definition 33: The machine learning system of Definition 31, wherein the trained processor is to define an inner surgical guidance guard rail and an outer surgical guidance guard rail based upon the second segmentation, wherein a surgical tool path is to lie between the inner surgical guidance guard rail and the outer surgical guidance guard rail.
Definition 34: The machine learning system of Definition 33, wherein the trained processor is further configured output a recommended cutting path for a surgical tool, the cutting path being contained between the inner surgical guidance guard rail and the outer surgical guidance guard rail.
Definition 35: The machine learning system of Definition 33, wherein the trained processor is further configured to:
   define a segmentation buffer zone; and
   segment non-targeted features within the segmentation buffer zone.
Definition 36: The machine learning system of Definition 35, wherein the trained processor is further configured to adjust the outer surgical guidance guard rail based on segmented non-targeted features within the segmentation buffer zone.
Definition 37: The machine learning system of Definition 36, wherein the trained processor is further configured output a recommended cutting path for a surgical tool, the cutting path being contained between the inner surgical guidance guard rail and the outer surgical guidance guard rail.
Definition 38: The machine learning system of Definition 35, wherein the trained processor is further configured output a notice indicating regions between the inner surgical guidance guard rail and the outer surgical guidance guard rail where segmented non-targeted features are located.
Definition 39: The machine learning system of Definition 35, wherein the trained processor comprises:
   a first vision transformer to segment the non-linear outer surface of the volumetric mass of the targeted feature based upon real-time image data; and
   a second vision transformer to segment the non-targeted features within the segmentation buffer zone.
Definition 40: The machine learning system of Definition 35, wherein the targeted feature comprises a tumor.
Definition 41: The machine learning system of Definition 35, wherein the segmentation buffer zone comprises an inner boundary and an outer boundary and wherein the refinement zone lie between the inner boundary and the outer boundary.
Definition 42: The machine learning system of Definition 35, wherein the segmentation buffer zone comprises an inner boundary and an outer boundary, the inner boundary coinciding with the refinement zone.
Definition 43: The machine learning system of Definition 35 further comprising an inner surgical guard rail and an outer surgical guard rail, wherein the inner surgical guard rail and the outer surgical guard rail lie between the refinement zone and the outer boundary of a segmented buffer zone.
Definition 44: The machine learning system of Definition 35 further comprising an inner surgical guard rail and an outer surgical guard rail, wherein the outer surgical guard rail coincides with an outer boundary of the segmentation buffer zone.
Definition 45: The machine learning system of Definition 35 further comprising an inner surgical guard rail and an outer surgical guard rail, wherein the outer surgical guard rail is nonuniformly spaced from the inner surgical guard rail.
Definition 46: The machine learning system of Definition 35, wherein the outer surgical guard rail is shaped to exclude at least one of the segmented non-targeted features from between the inner surgical guard rail and the outer surgical guard rail.
Definition 47: The machine learning system of Definition 31, wherein the trained processor is configured to segment at least one of nerves and arteries within the segmentation buffer zone.
Definition 48: The machine learning system of Definition 31, wherein the segmentation buffer zone has a nonuniform width about the refinement zone.
Definition 49: The machine learning system of Definition 31, wherein the trained processor is configured to segment a first portion of the segmentation buffer zone at a first resolution and to segment a second portion of the segmentation buffer zone at a second resolution greater than the first resolution.
Definition 50: The machine learning system of Definition 31 further comprising a display, wherein the trained processor is configured to concurrently present boundaries of the segmentation buffer zone, with the segmented portion, and those features segmented in the segmentation buffer zone, on the display.
Definition 51: The machine learning system of Definition 31, wherein the trained processor is to define a width of the segmentation buffer zone based upon the refinement zone.
Definition 52: The machine learning system of Definition 31, wherein the trained processor is trained to classify the targeted feature and to define a width of the segmentation buffer zone based upon the classification of the targeted feature.
Definition 53: The artificial system of Definition 31, wherein the trained processor is trained to classify the non-targeted features and to differently segment the non-targeted features based upon their classification.
Definition 54: The machine learning system of Definition 31, wherein the trained processor is configured to segment the non-linear outer surface of the volumetric mass of the targeted feature by successively applying different algorithms to smaller and smaller portions of the real-time image data, each of the different successive algorithms having a smaller down sampling of image data.
Definition 55: A machine learning system for guiding a surgical tool, the system comprising a trained processor configured to:
   receive a machine trained model trained on training images comprising inner and outer surgical guard rails for a surgical procedure; and:
   define an inner surgical guidance guard rail and an outer surgical guidance guard rail on a real-time ultrasound image, the inner surgical guidance guard rail and an outer surgical guidance guard rail being based upon the machine trained model, wherein a surgical tool path is to lie between the inner surgical guidance guard rail and the outer surgical guidance guard rail.
Definition 56: The machine learning system of Definition 55, wherein the trained processor is further configured output a recommended cutting path for a surgical tool, the cutting path being contained between the inner surgical guidance guard rail and the outer surgical guidance guard rail.
Definition 57: A machine learning system for real-time segmentation of clinically relevant features during a surgical procedure, the system comprising:
   a trained processor to:
      perform a segmentation of a non-linear outer surface of a volumetric mass of a targeted feature based upon real-time volumetric ultrasound data at a first resolution; and
      define an inner surgical guidance guard rail and an outer surgical guidance guard rail based upon the segmentation, wherein a surgical tool path is to lie between the inner surgical guidance guard rail and the outer surgical guidance guard rail.
Definition 58: A tumor segmentation system comprising:
   a processor; and
   a non-transitory computer readable medium containing instructions to direct the processor to:
      receive source images of an anatomy;
      generate synthetic ultrasound images of the anatomy;
      train an artificial intelligence machine learning network to learn locations of the tumor surface based on synthetic ultrasound images;
      determine a confidence band based on the locations;
      define a refinement zone in a real-time ultrasound image based on the confidence band; and
      segment a tumor surface in a real-time ultrasound image in the refinement zone.
Definition 59: The tumor segmentation system of Definition 58, wherein the synthetic ultrasound images of the anatomy are based upon a physics based synthetic model.
Definition 60: The tumor segmentation system of Definition 58, wherein the synthetic ultrasound images are based upon a source image comprising chromatography (CT) scans.
Definition 61: The tumor segmentation system of Definition 58, wherein the synthetic ultrasound images are based upon a source image comprising ultrasound scans.
Definition 62: The tumor segmentation system of Definition 58, wherein the instructions to direct the processor to segment the tumor surface are based upon network training that is based upon synthetic ultrasound images depicting different tumors.
Definition 63: The tumor segmentation system of Definition 58, wherein the instructions are to direct the processor to segment features external to the tumor surface.
Definition 64: The tumor segmentation system of Definition 63, wherein the instructions are to direct the processor to segment a first type of feature and a second type of feature, and wherein the instructions are to further direct the processor to segment the first type of feature at a first resolution and the second type of feature at a second resolution different than the first resolution.
Definition 65: The tumor segmentation system of Definition 58, wherein the features external to the tumor surface comprise nerves.
Definition 66: The tumor segmentation system of Definition 65, wherein the instructions to direct the processor to segment nerves external to the tumor surface are based upon network training that is based upon synthetic ultrasound images depicting different nerves proximate tumor surfaces.
Definition 67: The tumor segmentation system of Definition 58, wherein the features external to the tumor surface comprise arteries.
Definition 68: The tumor segmentation system of Definition 67, wherein the instructions to direct the processor to segment nerves external to the tumor surface are based upon network training that is based upon synthetic ultrasound images depicting different arteries proximate tumor surfaces.
Definition 69: The tumor segmentation system of Definition 58, wherein the instructions are to direct the processor to segment a first portion of the tumor surface at a first resolution and to segment a second portion of the tumor surface at a second resolution different than the first resolution.
Definition 70: A machine learning system for real-time segmentation of clinically relevant features during a surgical procedure, the system comprising:
   a trained processor to:
      determine presence of a non-targeted feature in a real-time ultrasound image; and
      in response to determining presence of the non-targeted feature, initiating segmentation of the non-targeted feature.
Definition 71: A machine learning system comprising:
   a trained processor to perform a segmentation of a clinically relevant feature in an ultrasound image during a surgical procedure in real time; and
      a display to present, in real time, a depiction of the clinically relevant feature based on the segmentation.

Although the present disclosure has been described with reference to example implementations, workers skilled in the art will recognize that changes may be made in form and detail without departing from the disclosure, For example, although different example implementations may have been described as including features providing various benefits, it is contemplated that the described features may be interchanged with one another or alternatively be combined with one another in the described example implementations or in other alternative implementations. Because the technology of the present disclosure is relatively complex, not all changes in the technology are foreseeable. The present disclosure described with reference to the example implementations and set forth in the following s is manifestly intended to be as broad as possible. For example, unless specifically otherwise noted, the claims reciting a single particular element also encompass a plurality of such particular elements. The terms “first”, “second”, “third” and so on in the claims merely distinguish different elements and, unless otherwise stated, are not to be specifically associated with a particular order or particular numbering of elements in the disclosure.

What is claimed is:

1. A system for real-time segmentation of clinically relevant features during a surgical procedure, the system comprising:
a trained processor to:
perform a first segmentation of a real-time ultrasound image depicting a non-linear outer surface of a volumetric mass of a targeted feature based upon real-time ultrasound data at a first resolution;
define a refinement zone based upon the first segmentation; and
perform a second segmentation in the refinement zone of the real-time ultrasound image at a second resolution greater than the first resolution.

2. The system of claim 1, wherein the refinement zone comprises an inner boundary and an outer boundary and wherein the outer surface of the targeted feature lies between the inner boundary and the outer boundary of the refinement zone.

3. The system of claim 1, wherein the trained processor is to define an inner surgical guidance guard rail and an outer surgical guidance guard rail based upon the second segmentation, wherein a surgical tool path is to lie between the inner surgical guidance guard rail and the outer surgical guidance guard rail.

4. The system of claim 3, wherein the trained processor is further configured output a recommended cutting path for a surgical tool, the cutting path being contained between the inner surgical guidance guard rail and the outer surgical guidance guard rail.

5. The system of claim 3, wherein the trained processor is further configured to:
define a segmentation buffer zone; and
segment non-targeted features within the segmentation buffer zone.

6. The system of claim 5, wherein the trained processor is further configured to adjust the outer surgical guidance guard rail based on segmented non-targeted features within the segmentation buffer zone.

7. The system of claim 6, wherein the trained processor is further configured to output a recommended cutting path for a surgical tool, the cutting path being contained between the inner surgical guidance guard rail and the outer surgical guidance guard rail.

8. The system of claim 5, wherein the trained processor is configured to segment a first portion of the segmentation buffer zone at a first resolution and to segment a second portion of the segmentation buffer zone at a second resolution greater than the first resolution.

9. The system of claim 5, further comprising a display, wherein the system is configured to concurrently present boundaries of the segmentation buffer zone with those features segmented in the segmentation buffer zone, on the display.

10. The system of claim 5, wherein the trained processor is further configured to output a notice indicating regions between the inner surgical guidance guard rail and the outer surgical guidance guard rail where segmented non-targeted features are located.

11. The system of claim 5, wherein the trained processor is to define a width of the segmentation buffer zone based upon the refinement zone.

12. The system of claim 5, wherein the trained processor is trained to classify the targeted feature and to define a width of the segmentation buffer zone based upon the classification of the targeted feature.

13. The system of claim 1, wherein the trained processor is trained to classify non-targeted features and to segment the non-targeted features based upon their classification.

14. The system of claim 1, wherein the trained processor is configured to segment the non-linear outer surface of the volumetric mass of the targeted feature by successively applying different algorithms to smaller and smaller portions of the real-time volumetric ultrasound data, each of the different successive algorithms having a smaller down sampling of ultrasound data.

15. The system of claim 1, wherein the trained processor is configured to:
perform a third segmentation of a cutting tool path segmentation zone about a cutting tool path from a surface of an organ to the targeted feature within the organ based upon real-time ultrasound data at a third resolution;
identify a non-targeted feature proximate the cutting tool path based on the third segmentation; and
modify the cutting tool path based on the identified non-targeted feature.

16. The system of claim 1, wherein the trained processor comprises one of more networks.
